(19) United States
(12) Patent Application Publication
(10) Pub. No.: US 2011/0237612 A1
(43) Pub. Date: Sep. 29, 2011

(54) Thienylamino pyrimidines for use as Fungicides

(75) Inventors: Jörg Nico Greul, Leverkusen (DE); Hendrik Helmke, Liederbach (DE); Stefan Hillebrand, Neuss (DE); Amos Mattes, Langenfeld (DE); Carl Friedrich Nising, Langenfeld (DE); Pierre Wasnaire, Dusseldorf (DE); Ulrike Wachendorff-Neumann, Neuwied (DE); Peter Dahmen, Neuss (DE); Arnd Voerste, Koln (DE); Ruth Meissner, Leverkusen (DE); Christoph Andreas Braun, Dusseldorf (DE); Martin Kaussmann, Koln (DE); Hiroyuki Hadano, Tochigi (JP)

(73) Assignee: BayerCropScience AG, 40789 Monheim (DE)

(21) Appl. No.: 13/061,930

(22) PCT Filed: Aug. 22, 2009
(86) PCT No.: PCT/EP2009/006116

§ 371 Date: May 27, 2011

(30) Foreign Application Priority Data
Sep. 3, 2008 (EP) 08163560.9

(51) Int. Cl. A01N 43/54; C07D 409/12; C07D 239/48; A01P 3/00

(52) U.S. Cl. 514/275; 544/324; 544/323

(57) ABSTRACT

Thienylaminopyrimidines of the formula (I)

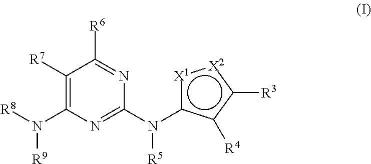

in which to R¹ to R¹⁰ and X¹ and X² have the meanings given in the description, and agrochemically active salts thereof, their use and also methods and compositions for controlling phytopathogenic harmful fungi in and/or on plants or in and/or on seed of plants, processes for preparing such compositions and treated seed and also their use for controlling phytopathogenic harmful fungi in agriculture, horticulture and forestry, in the protection of materials and in the domestic and hygiene field. The present invention furthermore relates to a process for preparing thienylaminopyrimidines of the formula (I).

The invention relates to thienylaminopyrimidines and their agrochemically active salts, to their use and to methods and compositions for controlling phytopathogenic harmful fungi in and/or on plants or in and/or on seed of plants, to processes for preparing such compositions and treated seed and also to their use for controlling phytopathogenic harmful fungi in agriculture, horticulture and forestry, in the protection of materials and in the domestic and hygiene field. The present invention furthermore relates to a process for preparing thienylaminopyrimidines.

Since the ecological and economic demands made on modern crop protection agents are increasing constantly, for example with respect to activity spectrum, toxicity, selectivity, application rate, formation of residues and favourable manufacture, and there can furthermore be problems, for example, with resistance, there is a constant need to develop novel fungicides which, at least in some areas, have advantages over the known fungicides.

Surprisingly, it has now been found that the present thiophene-substituted aminopyrimidines solve at least some aspects of the objects mentioned and are suitable for use as crop protection agents, in particular as fungicides.

Sulphonamide-substituted thiopheneaminopyrimidines are already known as pharmaceutically active compounds (see, for example, WO 03/076437), but not their surprising fungicidal activity.

The invention provides compounds of the formula (I)

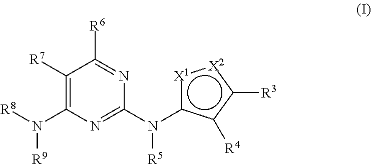

in which one or more of the symbols have one of the meanings below:
X¹ represents sulphur or CR¹,
X² represents sulphur or CR²,
   where exactly one of the radicals X¹ and X² represents a sulphur atom,
R¹ represents hydrogen, C₁-C₄-alkyl, C₁-C₄-alkoxy or halogen,
   if X² represents CR²,
R² and R³ independently of one another represent hydrogen, halogen, CN, nitro, OR¹⁰, O(CH₂)ₘOR¹⁰, O[C(R¹⁰)₂]ₘOR¹⁰, O[C(R¹⁰)₂]ₘN(R¹⁰)₂, OCOR¹¹, SR¹⁰, SOR¹⁰, SO₂R¹⁰, C═OR¹⁰, CH═NOR¹⁰, CR¹¹═NOR¹⁰, COCl, CON(R¹⁰)₂, COOR¹⁰, NR¹⁰COR¹⁰, N(R¹⁰)₂, [C(R¹⁰)₂]ₘCN, (CH₂)ₘOR¹⁰, (CH₂)ₘR¹⁰, [C(R¹⁰)₂]ₘSR¹⁰, (CH₂)ₘSOR¹⁰, (CH₂)ₘSO₂R¹⁰, (CH₂)ₘN(R¹⁰)₂, [C(R¹⁰)₂]ₘN(R¹⁰)₂, (CH₂)ₘCOR¹⁰, [C(R¹⁰)₂]ₘOR¹⁰, [C(R¹⁰)₂]ₘCOR¹⁰, unsubstituted or substituted C₁-C₈-alkyl, C₂-C₆-alkenyl or C₁-C₈-haloalkyl; where m=1-4,
   where the substituents independently of one another are selected from the group consisting of:
   hydrogen, fluorine, chlorine or bromine, C₁-C₄-alkyl, C₁-C₄-alkoxy, hydroxyl, oxo, C₁-C₄-haloalkyl and cyano,
      where, if X² represents a sulphur atom, the above definitions apply only to R³,
R⁴ represents hydrogen, C₁-C₄-alkyl, C₁-C₄-alkoxy or halogen,
R⁵ represents hydrogen, C₁-C₂-alkyl, C₁-C₄-alkoxy-C₁-C₄-alkyl, C₁-C₄-trialkylsilyl, C₁-C₄-trialkylsilylethyl, C₁-C₄-dialkylmonophenylsilyl, CHO, (C₁-C₄-alkyl)carbonyl, (C₁-C₄-alkoxy-C₁-C₄-alkyl)carbonyl, (C₃-C₆-alkenyloxy)carbonyl, (C₃-C₆-cycloalkyl)carbonyl, (halo-C₁-C₄-alkoxy-C₁-C₄-alkyl)carbonyl, (C₁C₄-haloalkyl)carbonyl, (C₁-C₄-alkoxy)carbonyl, (C₁-C₄-haloalkoxy)carbonyl, benzyloxycarbonyl, unsubstituted or substituted benzyl, unsubstituted or substituted C₂-C₆-alkenyl, unsubstituted or substituted C₂-C₆-alkynyl, C₁-C₂-alkylsulfinyl or C₁-C₂-alkylsulphonyl, where the substituents independently of one another are selected from the group consisting of:
   hydrogen, halogen, nitro, C₁-C₄-alkyl, C₁-C₄-alkoxy, hydroxyl, C₁-C₄-haloalkyl or cyano,
R⁶ represents hydrogen, C₁-C₃-alkyl, cyano or C₁-C₃-haloalkyl,
R⁷ represents halogen, cyano, C₁-C₃-alkyl, C₁-C₃-haloalkyl, C₁-C₃-haloalkyloxy, SMe, SOMe or SO₂Me,
R⁸ represents hydrogen, C₁-C₂-alkyl, C₁-C₄-alkoxy-C₁-C₄-alkyl, C₁-C₆-trialkylsilyl, C₁-C₄-trialkylsilylethyl, C₁-C₄-dialkylmonophenylsilyl, (C₁-C₄-alkyl)carbonyl, (C₁-C₄-haloalkyl)carbonyl, (C₁-C₄-alkoxy)carbonyl, unsubstituted or substituted benzyl, unsubstituted or substituted C₂-C₆-alkenyl, unsubstituted or substituted C₂-C₆-alkynyl, C₁-C₆-alkylsulphinyl, C₁-C₆-alkylsulphonyl, C₁-C₆-haloalkylsulphinyl or C₁-C₆-haloalkylsulphonyl,
   where the substituents independently of one another are selected from the group consisting of:
   fluorine, chlorine and bromine atoms, cyano, hydroxyl, methoxy and CF₃,
R⁹ represents straight-chain or branched unsubstituted or substituted C₁-C₇-alkyl, straight-chain or branched unsubstituted or substituted C₂-C₇-haloalkyl, unsubstituted or substituted C₃-C₇-cycloalkyl, straight-chain or branched unsubstituted or substituted C₃-C₇-cycloalkyl-C₁-C₃-alkyl, straight-chain or branched unsubstituted or substituted C₃-C₇-alkenyl, straight-chain or branched unsubstituted or substituted C₃-C₇-alkynyl, straight-chain or branched unsubstituted or substituted C₁-C₄-alkoxy-C₁-C₄-alkyl, straight-chain or branched unsubstituted or substituted C₁-C₄-haloalkoxy-C₁-C₄-alkyl, 2-methyl-1-(methylsulphanyl)propan-2-yl or oxetan-3-yl,
   or
   R⁸ and R⁹ together with the nitrogen atom to which they are attached form an unsubstituted or substituted 3- to 7-membered saturated cycle which may contain up to one further heteroatom selected from the group consisting of oxygen, sulphur and nitrogen,
R¹⁰ are identical or different and represent hydrogen, C₁-C₆-alkyl, C₁-C₆-haloalkyl, unsubstituted or substituted C₃-C₆-cycloalkyl, C₁-C₄-trialkylsilyl, unsubstituted or substituted C₂-C₄-alkenyl, unsubstituted or substituted C₂-C₄-alkynyl, unsubstituted or substituted phenyl, C₁-C₄-alkoxy-C₁-C₄-alkyl, C₁-C₄-alkylthio-C₁-C₄-alkyl, unsubstituted or substituted benzyl or a 3- to 7-membered unsubstituted or substituted saturated or unsaturated cycle which may contain no or up to four heteroatoms selected from the group consisting of N, O and S, where two oxygen atoms are not adjacent,
   or,
   if two radicals R¹⁰ are attached to a nitrogen atom, two radicals R¹⁰ may form a 3- to 7-membered unsubstituted or substituted saturated or unsaturated cycle which may contain up to four further heteroatoms selected from the group consisting of N, O and S, where two oxygen atoms are not adjacent,
   or
   if two radicals R¹⁰ are adjacent in the grouping NR¹⁰COR¹⁰, two radicals R¹⁰ may form a 3- to 7-membered unsubstituted or substituted saturated or unsaturated cycle which may contain up to four further heteroatoms selected from the group consisting of N, O and S, where two oxygen atoms are not adjacent,
R¹¹ are identical or different and represent C₁-C₈-alkyl, C₁-C₈-haloalkyl, C₁-C₄-trialkylsilyl, unsubstituted or substituted C₂-C₆-alkenyl, unsubstituted or substituted C₂-C₆-alkynyl, unsubstituted or substituted C₃-C₆-cycloalkyl, unsubstituted or substituted aryl, C₁-C₄-alkoxy-C₁-C₄-alkyl, unsubstituted or substituted benzyl or a 3- to 7-membered unsubstituted or substituted saturated or unsaturated cycle which may contain no or up to four heteroatoms selected from the group consisting of N, O and S, where two oxygen atoms are not adjacent,
   where the substituents in R¹⁰ independently of one another are selected from the group consisting of:
   methyl, ethyl, isopropyl, cyclopropyl, fluorine, chlorine and bromine atoms, methoxy, ethoxy, methylmercapto, ethylmercapto, cyano, hydroxyl and CF₃,
      and also agrochemically active salts thereof.

The invention also provides the use of the compounds of the formula (I) as fungicides.

The thienylaminopyrimidines of the formula (I) according to the invention and their agrochemically active salts are highly suitable for controlling phytopathogenic harmful fungi. The compounds according to the invention mentioned above have in particular fungicidal activity and can be used both in crop protection, in the domestic and hygiene field and in the protection of materials.

The compounds of the formula (I) can be present both in pure form and as mixtures of various possible isomeric forms, in particular of stereoisomers, such as E and Z, threo and erythro, and also optical isomers, such as R and S isomers or atropisomers, and, if appropriate, also of tautomers. What is claimed are both the E and the Z isomers, and the threo and erythro, and also the optical isomers, mixtures of these isomers, and also the possible tautomeric forms.

Preference is given to compounds of the formula (I) in which one or more of the symbols have one of the meanings below:
X¹ represents sulphur or CR¹,
X² represents sulphur or CR²,
   where exactly one of the radicals X¹ and X² represents a sulphur atom,
R¹ represents hydrogen, methyl, methoxy or Cl,
   if X² represents CR²,
R² and R³ independently of one another represent hydrogen, halogen, CN, nitro, hydroxyl, O—C₁-C₄-alkyl, O—(C₁-C₃-haloalkyl), O—(C₃-C₆-cycloalkyl), O—C₂-C₄-alkenyl, O—C₂-C₄-alkynyl, O(CH₂)ₘ, O(C₁-C₄-alkyl), OPh, OCO(C₁-C₄-alkyl), SH, S—C₁-C₄-alkyl, S—C₁-C₃-haloalkyl, SPh, SO(C₁-C₄-alkyl), SO₂(C₁-C₄-alkyl), SO₂(C₁-C₃-haloalkyl), SO₂(C₂-C₄-alkenyl), SO₂(C₂-C₄-alkynyl), CHO, CO(C₁-C₄-alkyl), CH═NO(C₁-C₄-alkyl), C(C₁-C₄-alkyl)=NO(C₁-C₄-alkyl), CONH(C₁-C₄-alkyl), CON(C₁-C₄-alkyl)₂, CON(SiMe₃)₂, CONH(C₁-C₃-haloalkyl), CONH(C₂-C₄-alkenyl), CONH(C₂-C₄-alkynyl), CONH(C₃-C₆-cycloalkyl), CONHCH₂C(═CH₂)CH₃, CONHCH(CH₃)CH₂O(C₁-C₄-alkyl), CONH(CH₂)ₘO(C₁-C₄-alkyl), CONH(CH₂)ₘS(C₁-C₄-alkyl), CONHCH(CH₃)CH₂S(C₁-C₄-alkyl), CONHPh, pyrrolidin-1-ylcarbonyl, piperidin-1-ylcarbonyl, (4-methylpiperazin-1-yl)carbonyl, azetidin-1-ylcarbonyl, aziridin-1-ylcarbonyl, hexamethylenimin-1-yl-carbonyl, morpholin-1-ylcarbonyl, thiomorpholin-1-ylcarbonyl, COOH, COCl, (C₁-C₄-alkoxy)carbonyl, NHCO(C₁-C₄-alkyl), NHCO(C₁-C₄-haloalkyl), N(C₁-C₂-alkyl)CO(C₁-C₄-alkyl), NHCO(C₂-C₄-alkenyl), NHCOPh, NHCO(C═CH₂)CH₃, NHCON(C₁-C₄-alkyl)₂, NHCO(CH₂)ₘO(C₁-C₄-alkyl), NHCHO, N(C₁-C₄-alkyl)CHO, NH₂, NH(C₁-C₄-alkyl), N(C₁-C₄-alkyl)₂, NHCH(C₁-C₄-alkyl)CH₂O(C₁-C₄-alkyl), (CH₂)ₘCN, (CH₂)ₘSO(C₁-C₄-alkyl), (CH₂)ₘSO₂(C₁-C₄-alkyl), (CH₂)ₘCO(C₁-C₄-alkyl), (CH₂)ₘO(C₁-C₄-alkyl), C(CH₃)₂O(C₁-C₄-alkyl), (CH₂)ₘC(C₁-C₄-alkyl)₂O(C₁-C₄-alkyl), (CH₂)OH, (CH₂)ₘS(C₁-C₄-alkyl), C(CH₃)₂S(C₁-C₄-alkyl), (CH₂)ₘNH(C₁-C₄-alkyl), (CH₂)ₘN(C₁-C₄-alkyl)₂, C₁-C₅-alkyl, C₃-C₆-cycloalkyl or C₁-C₃-haloalkyl,
   where, if X² represents a sulphur atom, the above definition applies only to R³ and m corresponds to a number from 1 to 4,
R⁴ represents hydrogen, methyl, methoxy, chlorine or fluorine,
R⁵ represents hydrogen, Me, COMe, CHO, COCH₂OCH₃, CH₂OCH₃, COOMe, COOEt, COOtertBu, COOBn, COCF₃, CH₂CH═CH₂, CH₂CCH, SOCH₃, SO₂CH₃ or benzyl,
R⁶ represents hydrogen, cyano, methyl, CF₃ or CFH₂,
R⁷ represents fluorine, chlorine, bromine, iodine, methyl, OCF₃ or CF₃,
R⁸ represents hydrogen, methyl, ethyl, propyl, propan-2-yl, 2-methoxyethan-1-yl, prop-2-en-1-yl, CH₂OCH₃, COH, COMe, COOMe, COOEt, COOtertBu, COCF₃ or benzyl,
R⁹ represents straight-chain or branched unsubstituted or substituted C₁-C₅-alkyl, unsubstituted or substituted C₃-C₆-cycloalkyl, straight-chain or branched unsubstituted or substituted C₃-C₆-cycloalkyl-C₁-C₂-alkyl, unsubstituted or substituted C₂-C₅-haloalkyl, straight-chain or branched unsubstituted or substituted C₃-C₅-alkenyl or 2-methyl-1-(methylsulphanyl)propan-2-yl,
   where the substituents in R⁹ independently of one another are selected from the group consisting of methyl, ethyl, isopropyl, cyclopropyl, fluorine, chlorine and bromine atoms, methoxy, ethoxy, methylmercapto, ethylmercapto, cyano, hydroxyl and CF₃,
   and also agrochemically active salts thereof.

Particular preference is given to compounds of the formula (I) in which one or more of the symbols have one of the meanings below:
X¹ represents sulphur or CR¹,
X² represents sulphur or CR²,
   where exactly one of the radicals X¹ and X² is a sulphur atom,
R¹ represents hydrogen,
   if X² represents CR²,
R² and R³ independently of one another represent hydrogen, COOMe, COOEt, COOPr, COOiPr, CONH(C₄H₉), CONH(CH₂)₂OMe, CONHCH(CH₃)CH₂OMe, CONHOH, CONHMe, CONHEt, CONHPr, CONHiPr, CONH(i-C₄H₉), CONHPh, CONH(CH₂)₂SCH₃, CONHCH(CH₃)CH₂SCH₃, CONHCH₂CF₃, CONHCH₂CH═CH₂, CONHCH₂C≡H, CONMeCH₂CCH, CONHCH₂C(═CH₂)CH₃, CONHPh, CONHcyclopropyl, CON(Me)iC₃H₇, CON(Me)CH₂CH═CH₂, CON(Et)₂, CON(Me)cyclopropylmethyl, CON(Me)cyclobutylmethyl, CON(Ph)₂, CON(Me)₂, CON(Pr)₂, pyrrolidin-1-ylcarbonyl, piperidin-1-ylcarbonyl, (4-methylpiperazin-1-yl)carbonyl, azetidin-1-ylcarbonyl, aziridin-1-yl-carbonyl, hexamethylenimin-1-ylcarbonyl, morpholin-1-ylcarbonyl, thiomorpholin-1-ylcarbonyl, CON(SiMe₃)₂, COMe, COEt, COPr, CN, C(═NOCH₃)Me, C(═NOEt)Me, C(═NOPr)Me, chlorine, bromine, iodine, nitro, SH, SMe, SEt, SPr, SCF₃, SPh, COOH, Me, Et, Pr, SO₂Me, SO₂Et, CH₂OMe or CH₂OEt,
   where, if X² represents a sulphur atom, the above definition applies only to R³,
R⁴ represents hydrogen,
R⁵ represents hydrogen, COMe, CHO, CH₂OCH₃, COOMe or CH₂CCH,
R⁶ represents hydrogen,
R⁷ represents fluorine, chlorine, bromine, iodine, OCF₃ or CF₃,
R⁸ represents hydrogen or methyl,
R⁹ represents cyclopropyl, cyclobutyl, 2-methylcycloprop-1-yl, 2-methylcyclobut-1-yl, 3-methylcyclobut-1-yl, 2,2-difluoroethyl, 2,2,2-trifluoroethyl, isopropyl, cyclopropylmethyl, methyl, ethyl, 2,2-dimethylcyclopropyl, cyclopentyl, propan-2-yl, prop-2-en-1-yl, butan-2-yl, 1-methoxypropan-2-yl, 2-methyl-1-(methylsulphanyl)propan-2-yl, oxetan-3-yl, 1,1,1-trifluoropropan-2-yl or 2,2,3,3,3-pentafluoropropyl,
   and also agrochemically active salts thereof.

Very particular preference is given to compounds of the formula (I) in which one or more of the symbols have one of the meanings below:
X¹ represents sulphur or CR¹,
X² represents sulphur or CR², where exactly one of the radicals X¹ and X² represents a sulphur atom,
R¹ represents hydrogen,
   if X² represents CR²,
R² and R³ independently of one another represent hydrogen, CH₃, COOMe, CONH(tert-C₄H₉), CONH(CH₂)₂OMe, CONHCH(CH₃)CH₂OMe, CONHOH, CON(Me)iC₃H₇, CON(Me)CH₂CH═CH₂, CON(Et)₂, CONmethylcyclopropylmethyl, pyrrolidin-1-ylcarbonyl, piperidin-1-ylcarbonyl, morpholin-1-ylcarbonyl, COMe, CN, C(═NOCH₃)CH₃, chlorine, bromine, SMe, CONHCH₂CF₃, CONHCH₂CH═CH₂, CONHCH₂CCH, CONMeCH₂CCH, CONHCH₂C(═CH₂)CH₃, CONHPh, CONHcyclopropyl, (4-methylpiperazin-1-yl)carbonyl, COOH or SO₂Me,
   where, if X² represents a sulphur atom, the above definition applies only to R³,
R⁴ represents hydrogen,
R⁵ represents hydrogen,
R⁶ represents hydrogen,
R⁷ represents chlorine, bromine or CF₃,
R⁸ represents hydrogen,
R⁹ represents cyclopropyl, cyclobutyl, 2-methylcycloprop-1-yl, 2-methylcyclobut-1-yl, 3-methylcyclobut-1-yl, 2,2-difluoroethyl, 2,2,2-trifluoroethyl, isopropyl or cyclopropylmethyl,
   and also agrochemically active salts thereof.

Very particular preference is furthermore given to compounds of the formula (I) in which one or more of the symbols have one of the meanings below:
X¹ represents sulphur,
X² represents CR²,
R² represents hydrogen, CH₃, COOMe, CONH(CH₂)₂OMe, COMe, SMe, SO₂Me or CN,
R³ represents hydrogen or CN,
   or
X¹ represents CR¹,
X² represents sulphur,
R¹ represents hydrogen,
R³ represents hydrogen, COOMe, CON(Me)(iC₃H₇), CONH(tert-C₄H₉), CONH(CH₂)₂OMe, CONHCH(CH₃)CH₂OMe, CONHOH, CON(Me)CH₂CH═CH₂, CONHCH₂CH═CH₂, CON(Et)₂, pyrrolidin-1-ylcarbonyl, piperidin-1-ylcarbonyl, morpholin-1-ylcarbonyl, COMe or CN,
R⁴ represents hydrogen,
R⁵ represents hydrogen,
R⁶ represents hydrogen,
R⁷ represents chlorine, bromine or CF₃,
R⁸ represents hydrogen,
R⁹ represents cyclopropyl, cyclobutyl, 2-methylcycloprop-1-yl, 2-methylcyclobut-1-yl, 3-methylcyclobut-1-yl, 2,2-difluoroethyl, 2,2,2-trifluoroethyl, isopropyl or cyclopropylmethyl,
   and also agrochemically active salts thereof.

Preference is furthermore given to compounds of the formula (I) in which
X¹ represents a sulphur atom,
   where the other substituents have one or more of the meanings mentioned above,
   and the agrochemically active salts thereof.

Preference is furthermore given to compounds of the formula (I) in which

X² represents a sulphur atom,

where the other substituents have one or more of the meanings mentioned above,
and the agrochemically active salts thereof.

Preference is furthermore given to compounds of the formula (I) in which
X² represents a sulphur atom, and
R¹ and R⁵ both represent hydrogen,
   where the other substituents have one or more of the meanings mentioned above,
   and the agrochemically active salts thereof.

Preference is furthermore given to compounds of the formula (I) in which
R² represents hydrogen, CH₃, COOMe, CONH(C₄H₉), CONH(CH₂)₂OMe, CONHCH(CH₃)CH₂OMe, CONHOH, CON(Me)iC₃H₇, CON(Me)CH₂CH═CH₂, CON(Et)₂, CONmethylcyclopropylmethyl, pyrrolidin-1-ylcarbonyl, piperidin-1-ylcarbonyl, morpholin-1-ylcarbonyl, COMe, CN, C(═NOCH₃)CH₃, chlorine, bromine, SMe, CONHCH₂CF₃, CONHCH₂CH═CH₂, CONHCH₂CCH, CONMeCH₂CCH, CONHCH₂C(═CH₂)CH₃, CONHPh, CONHcyclopropyl, (4-methylpiperazin-1-yl)carbonyl, COOH or SO₂Me,
   where the other substituents have one or more of the meanings mentioned above,
   and the agrochemically active salts thereof.

Preference is furthermore given to compounds of the formula (I) in which
R³ represents hydrogen, CH₃, COOMe, CONH(C₄H₉), CONH(CH₂)₂OMe, CONHCH(CH₃)CH₂OMe, CONHOH, CON(Me)iC₃H₇, CON(Me)CH₂CH═CH₂, CON(Et)₂, CONmethylcyclopropylmethyl, pyrrolidin-1-ylcarbonyl, piperidin-1-ylcarbonyl, morpholin-1-ylcarbonyl, COMe, CN, C(═NOCH₃)CH₃, chlorine, bromine, SMe, CONHCH₂CF₃, CONHCH₂CH═CH₂, CONHCH₂CCH, CONMeCH₂C≡H, CONHCH₂C(═CH₂)CH₃, CONHPh, CONHcyclopropyl, (4-methylpiperazin-1-yl)carbonyl, COOH or SO₂Me,
   where the other substituents have one or more of the meanings mentioned above,
   and the agrochemically active salts thereof.

Preference is furthermore given to compounds of the formula (I) in which
R⁴ represents hydrogen,
   where the other substituents have one or more of the meanings mentioned above,
   and the agrochemically active salts thereof.

Preference is furthermore given to compounds of the formula (I) in which
R⁵ represents hydrogen,
   where the other substituents have one or more of the meanings mentioned above,
   and the agrochemically active salts thereof.

Preference is furthermore given to compounds of the formula (I) in which
R⁶ represents hydrogen,
   where the other substituents have one or more of the meanings mentioned above,
   and the agrochemically active salts thereof.

Preference is furthermore given to compounds of the formula (I) in which
R⁷ represents Cl, Br or CF₃,
   where the other substituents have one or more of the meanings mentioned above,
   and the agrochemically active salts thereof.

Preference is furthermore given to compounds of the formula (I) in which
R⁸ represents hydrogen or methyl,
   where the other substituents have one or more of the meanings mentioned above,
   and the agrochemically active salts thereof.

Preference is furthermore given to compounds of the formula (I) in which
R⁹ represents cyclopropyl, cyclobutyl, 2-methylcycloprop-1-yl, 2-methylcyclobut-1-yl, 3-methylcyclobut-1-yl, 2,2-difluoroethyl, 2,2,2-trifluoroethyl, isopropyl or cyclopropylmethyl,
   where the other substituents have one or more of the meanings mentioned above,
   and the agrochemically active salts thereof.

Preference is furthermore given to compounds of the formula (I) in which
X¹ represents CR¹ and
R¹, R⁴, R⁵ and R⁶ represent hydrogen,
   where the other substituents have one or more of the meanings mentioned above,
   and the agrochemically active salts thereof.

The radical definitions given above can be combined with one another as desired. Moreover, individual definitions may not apply.

Examples of inorganic acids are hydrohalic acids, such as hydrogen fluoride, hydrogen chloride, hydrogen bromide and hydrogen iodide, sulphuric acid, phosphoric acid and nitric acid, and acidic salts, such as NaHSO₄ and KHSO₄. Suitable organic acids are, for example, formic acid, carbonic acid and alkanoic acids, such as acetic acid, trifluoroacetic acid, trichloroacetic acid and propionic acid, and also glycolic acid, thiocyanic acid, lactic acid, succinic acid, citric acid, benzoic acid, cinnamic acid, oxalic acid, alkylsulphonic acids (sulphonic acids having straight-chain or branched alkyl radicals of 1 to 20 carbon atoms), arylsulphonic acids or aryldisulphonic acids (aromatic radicals, such as phenyl and naphthyl, which carry one or two sulphonic acid groups), alkylphosphonic acids (phosphonic acids having straight-chain or branched alkyl radicals of 1 to 20 carbon atoms), arylphosphonic acids or aryldiphosphonic acids (aromatic radicals, such as phenyl and naphthyl, which carry one or two phosphonic acid radicals), where the alkyl and aryl radicals may carry further substituents, for example p-toluenesulphonic acid, salicylic acid, p-aminosalicylic acid, 2-phenoxybenzoic acid, 2-acetoxybenzoic acid, etc.

Suitable metal ions are in particular the ions of the elements of the second main group, in particular calcium and magnesium, of the third and fourth main group, in particular aluminium, tin and lead, and also of the first to eighth transition group, in particular chromium, manganese, iron, cobalt, nickel, copper, zinc and others. Particular preference is given to the metal ions of the elements of the fourth period. Here, the metals can be present in various valencies that they can assume.

Optionally substituted groups may be mono- or polysubstituted, where in the case of polysubstitution the substituents may be identical or different.

In the definitions of the symbols given in the formulae above, collective terms were used which are generally representative for the following substituents:

halogen: fluorine, chlorine, bromine and iodine;
aryl: an unsubstituted or optionally substituted 5- to 15-membered partially or fully unsaturated mono-, bi- or tricyclic ring system having up to 3 ring members selected from the groups C(═O), (C═S), where at least one of the rings of the ring system is fully unsaturated, such as, for example (but not limited thereto) benzene, naphthalene, tetrahydronaphthalene, anthracene, indane, phenanthrene, azulene;
alkyl: saturated straight-chain or branched hydrocarbon radicals having 1 to 10 carbon atoms, such as, for example (but not limited thereto) methyl, ethyl, propyl, 1-methylethyl, butyl, 1-methyl-propyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, heptyl, 1-methylhexyl, octyl, 1,1-dimethylhexyl, 2-ethylhexyl, 1-ethylhexyl, nonyl, 1,2,2-trimethylhexyl, decyl;
haloalkyl: straight-chain or branched alkyl groups having 1 to 4 carbon atoms (as mentioned above), where in these groups some or all of the hydrogen atoms may be replaced by halogen atoms as mentioned above, such as, for example (but not limited thereto), C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2-difluoroethyl, 2,2-dichloro-2-fluoroethyl, 2,2,2-trichloroethyl, pentafluoroethyl and 1,1,1-trifluoroprop-2-yl;
alkenyl: unsaturated straight-chain or branched hydrocarbon radicals having 2 to 16 carbon atoms and at least one double bond in any position, such as, for example (but not limited thereto), C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
alkynyl: straight-chain or branched hydrocarbon groups having 2 to 16 carbon atoms and at least one triple bond in any position, such as, for example (but not limited thereto), C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
alkoxy: saturated straight-chain or branched alkoxy radicals having 1 to 4 carbon atoms, such as, for example (but not limited thereto), C₁-C₄-alkoxy, such as methoxy, ethoxy, propoxy, 1-methylethoxy, butoxy, 1-methyl-propoxy, 2-methylpropoxy, 1,1-dimethylethoxy;
haloalkoxy: straight-chain or branched alkoxy groups having 1 to 4 carbon atoms (as mentioned above), where some or all of the hydrogen atoms in these groups may be replaced by halogen atoms as mentioned above, such as, for example (but not limited thereto), C₁-C₂-haloalkoxy, such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluorethoxy, 2,2-dichloro-2-fluorethoxy, 2,2,2-trichloroethoxy, pentafluoroethoxy and 1,1,1-trifluoroprop-2-oxy;
thioalkyl: saturated straight-chain or branched alkylthio radicals having 1 to 6 carbon atoms, such as, for example (but not limited thereto), C₁-C₆-alkylthio, such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio;
thiohaloalkyl: straight-chain or branched alkylthio groups having 1 to 6 carbon atoms (as mentioned above), where some or all of the hydrogen atoms in these groups may be replaced by halogen atoms as mentioned above, such as, for example (but not limited thereto) C₁-C₂-haloalkylthio, such as chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio, pentafluoroethylthio and 1,1,1-trifluoroprop-2-ylthio;
cycloalkyl: mono-, bi- or tricyclic saturated hydrocarbon groups having 3 to 10 carbon ring members, such as, for example (but not limited thereto), cyclopropyl, cyclobutyl, cyclopentyl and cyclohexyl, bicyclo[1,0,1]butane, decalinyl, norbornyl;
cycloalkenyl: mono-, bi- or tricyclic non-aromatic hydrocarbon groups having 5 to 15 carbon ring members and at least one double bond, such as, for example (but not limited thereto) cyclopenten-1-yl, cyclohexen-1-yl, cyclohepta-1,3-dien-1-yl, norbornen-1-yl;
(alkoxy)carbonyl: an alkoxy group having 1 to 4 carbon atoms (as mentioned above) which is attached to the skeleton via a carbonyl group (—CO—);
heterocyclyl: a three- to fifteen-membered saturated or partially unsaturated heterocycle which contains one to four heteroatoms from the group consisting of oxygen, nitrogen and sulphur: mono-, bi- or tricyclic heterocycles containing, in addition to carbon ring members, one to three nitrogen atoms and/or one oxygen or sulphur atom or one or two oxygen and/or sulphur atoms; if the ring contains a plurality of oxygen atoms, these are not directly adjacent; such as, for example (but not limited thereto), oxiranyl, aziridinyl, 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetra-hydropyranyl, 4-tetrahydropyranyl, 2-tetrahydrothienyl, 3-hexahydro-pyridazinyl, 4-hexa-hydropyridazinyl, 2-hexahydropyrimidinyl, 4-hexahydropyrimidinyl, 5-hexahydropyrimidinyl, 2-piperazinyl, 1,3,5-hexahydro-triazin-2-yl und 1,2,4-hexahydrotriazin-3-yl;
hetaryl: unsubstituted or optionally substituted, 5- to 15-membered partially or fully unsaturated mono-, bi- or tricyclic ring system where at least one of the rings of the ring system is fully unsaturated, comprising one to four heteroatoms from the group consisting of oxygen, nitrogen and sulphur, if the ring contains a plurality of oxygen atoms, these are not directly adjacent;
such as, for example (but not limited thereto),
5-membered heteroaryl which contains one to four nitrogen atoms or one to three nitrogen atoms and one sulphur or oxygen atom: 5-membered heteroaryl groups which, in addition to carbon atoms, may contain one to four nitrogen atoms or one to three nitrogen atoms and one sulphur or oxygen atom as ring members, for example 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,446-azol-2-yl;
benzo-fused 5-membered heteroaryl which contains one to three nitrogen atoms or one nitrogen atom and one oxygen or sulphur atom: 5-membered heteroaryl groups which, in addition to carbon atoms, may contain one to four nitrogen atoms or one to three nitrogen atoms and one sulphur or oxygen atom as ring members, and in which two adjacent carbon ring members or one nitrogen and one adjacent carbon ring member may be bridged by a buta-1,3-diene-1,4-diyl group in which one or two carbon atoms may be replaced by nitrogen atoms; for example benzindolyl, benzimidazolyl, benzothiazolyl, benzopyrazolyl, benzofuryl;
5-membered heteroaryl which contains one to four nitrogen atoms and is attached via nitrogen or benzo-fused 5-membered heteroaryl which contains one to three nitrogen atoms and is attached via nitrogen: 5-membered heteroaryl groups which, in addition to carbon atoms, may contain one to four nitrogen atoms or one to three nitrogen atoms as ring members and in which two adjacent carbon ring members or one nitrogen and one adjacent carbon ring member may be bridged by a buta-1,3-diene-1,4-diyl group in which one or two carbon atoms may be replaced by nitrogen atoms, where these rings are attached to the skeleton via one of the nitrogen ring members, for example 1-pyrrolyl, 1-pyrazolyl, 1,2,4-triazol-1-yl, 1-imidazolyl, 1,2,3-triazol-1-yl, 1,3,4-triazol-1-yl;
6-membered heteroaryl which contains one to three or one to four nitrogen atoms: 6-membered heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

Not included are combinations which contradict natural laws and which the person skilled in the art would therefore have excluded based on his expert knowledge. Excluded are, for example, ring structures having three or more adjacent oxygen atoms.

The present invention furthermore relates to a process for preparing the thienylaminopyrimidines of the formula (I) according to the invention, comprising at least one of steps (a) to (e) below:
(a) the reaction of 2,4-dihalopyrimidines of the formula (III) with amines of the formula (II) in the presence of a base, if appropriate in the presence of a solvent, if appropriate in the presence of a catalyst, to give compounds of the formula (V), according to the reaction scheme below (Scheme 1):

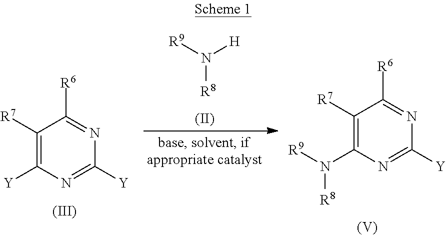

where Y═F, Cl, Br, I
(b) the reaction of compounds of the formula (V) with aminothiophenes of the formula (IV), if appropriate in the presence of an acid, if appropriate in the presence of a solvent, according to the reaction scheme below (Scheme 2):

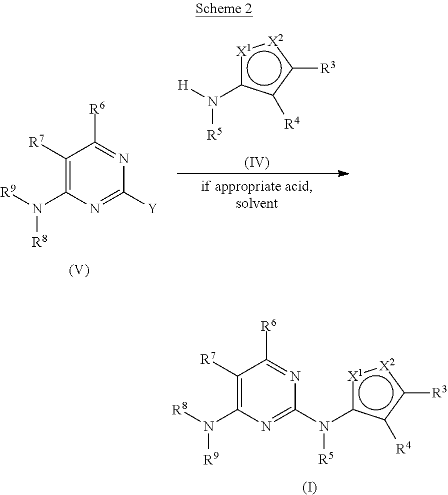

where Y═F, Cl, Br, I
(c) the reaction of compounds of the formula (VI) with an aminothiophene of the formula (IV), if appropriate in the presence of an acid and in the presence of a solvent, according to the reaction scheme below (Scheme 3):

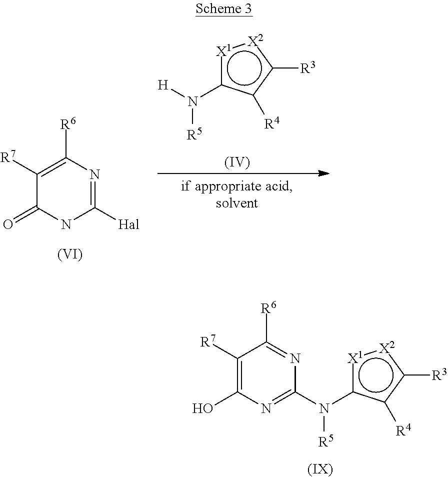

where Hal=F, Cl, Br, I
(d) the reaction of compounds of the formula (IX) with a halogenating agent, if appropriate in the presence of a solvent, to give compounds of the formula (X), according to the reaction scheme below (Scheme 4):

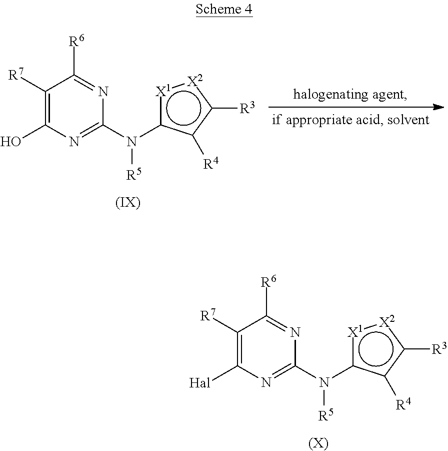

(e) the reaction of compounds of the formula (X) with amines of the formula (II) in the presence of a base, if appropriate in the presence of a solvent, if appropriate in the presence of a catalyst, to give compounds of the formula (I), according to the reaction scheme below (Scheme 5):

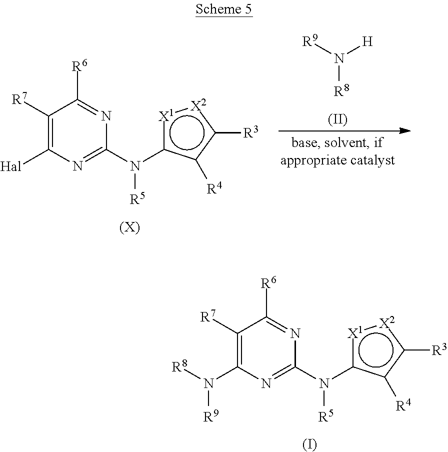

where the definitions of the radicals R¹ to R⁹ and X¹ and X² in the above schemes correspond to the given definitions and Y and Hal represent F, Cl, Br, I.

One way of preparing compounds of the formula (V) is shown in Scheme 1.

The alkylamino compounds of the formula (II) are either commercially available or can be prepared by literature procedures. One method for preparing suitable cyclopropylamino compounds of type (II) is, for example, the rearrangement of suitable carboxylic acid derivatives to the corresponding amino compounds (described, for example, in J. Am. Chem. Soc. 1961, 83, 3671-3678). Other methods, for example for preparing cyclobutylamino compounds of type (II), comprise the hydroboration of suitable cyclobutenes and subsequent treatment with NH₂SO₃H (for example Tetrahedron 1970, 26, 5033-5039), the reductive amination of cyclobutanones (described, for example, in J. Org. Chem. 1964, 29, 2588-2592) and also the reduction of nitro- or nitrosocyclobutanes (see, for example, J. Am. Chem. Soc. 1953, 75, 4044; Can. J. Chem. 1963, 41, 863-875) or azidocyclobutanes (described, for example, in Chem. Pharm. Bull. 1990, 38, 2719-2725; J. Org. Chem. 1962, 27, 1647-1650). The halogen-substituted amino compounds of the formula (II) are either commercially available or can be prepared by literature procedures. One method for preparing suitable halogen-substituted amino compounds (II) is, for example, the reduction of corresponding carboxamides (described, for example, in EP30092) or corresponding oximes or azides (described, for example, in Chem. Ber. 1988, 119, 2233) or nitro compounds (described, for example, in J. Am. Chem. Soc, 1953, 75, 5006). A further alternative consists in the treatment of corresponding aminocarboxylic acids with SF₄ in HF (described, for example, in J. Org. Chem. 1962, 27, 1406). The ring-opening of substituted aziridines with HF is described in J. Org. Chem. 1981, 46, 4938. Further methods for preparing halogen-substituted amino compounds (II) comprise the cleavage of corresponding phthalimides according to Gabriel (described, for example, in DE 3429048), the aminol ysis of suitable haloalkyl halides (described, for example, in US2539406) or the degradation of corresponding carboxylic acid azides (described, for example, in DE3611195). Using suitable fluorinating agents (for example DAST), aminoaldehydes or -ketones can be converted into the corresponding difluoroalkylamines (WO2008008022), whereas amino alcohols form the corresponding monofluoroalkylamines (for example WO2006029115). Analogously, using suitable chlorinating and brominating agents, chloro- and bromoalkylamines, respectively, can be obtained from amino alcohols (J. Org. Chem. 2005, 70, 7364, or Org. Lett., 2004, 6, 1935).

Suitable substituted 2,4-dihalopyrimidines (III) are either commercially available or can be prepared according to literature procedures, for example from commercially available substituted uracils (for example R⁷═CN: J. Org. Chem. 1962, 27, 2264; J. Chem. Soc. 1955, 1834; Chem. Ber. 1909, 42, 734; R⁷═CF₃: J. Fluorine Chem. 1996, 77, 93; see also WO 2000/047539).

Using a suitable base at a temperature of from −30° C. to +80° C. in a suitable solvent, such as, for example, dioxane, THF, dimethylformamide or acetonitrile, initially an amine (II) is reacted with a 2,4-dihalopyrimidine (III) over a period of 1-24 h. Suitable for the use as base are, for example, inorganic salts, such as NaHCO₃, Na₂CO₃ or K₂CO₃, organometallic compounds, such as LDA or NaHMDS, or amine bases, such as ethyldiisopropylamine, DBU, DBN or tri-n-butylamine. Alternatively, the reaction can also be carried out as described, for example, in Org. Lett. 2006, 8, 395 with the aid of a suitable transition metal catalyst, such as, for example, palladium, together with a suitiable ligand, such as, for example, triphenylphosphine or xanthphos.

Some of the compounds of the formula (V) are novel and thus also form part of the subject-matter of the present invention.

Novel are compounds of the formula (V) in which

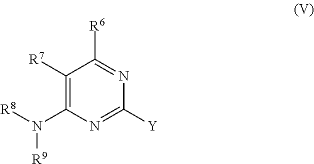

R⁶ represents hydrogen,
   and, if
R⁷ represents I, SMe, SOMe, SO₂Me, CF₃, CFH₂ or CF₂H, and
Y represents F, Cl, Br or I,
R⁸ represents hydrogen, ethyl, propyl, propan-2-yl, 2-methoxyethan-1-yl, prop-2-en-1-yl, CH₂OCH₃, COMe, COOMe, COOEt, COOtertBu, COCF₃ or benzyl, and
R⁹ represents cyclopropyl, cyclobutyl, 2-methylcycloprop-1-yl, 2-methylcyclobut-1-yl, 3-methylcyclobut-1-yl, 2,2-difluoroethyl, 2,2,2-trifluoroethyl, isopropyl, cyclopropylmethyl, methyl, ethyl, 2,2-dimethylcyclopropyl, cyclopentyl, propan-2-yl, prop-2-en-1-yl, butan-2-yl, 1-methoxypropan-2-yl, 2-methyl-1-(methylsulphanyl)propan-2-yl, 1,1,1-trifluoropropan-2-yl or 2,2,3,3,3-pentafluoropropyl.

Novel are compounds of the formula (V) in which

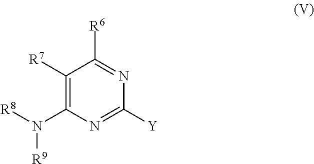

R⁶ represents hydrogen,
   and, if
R⁷ represents cyano, and
Y represents F, Cl, Br or I,
R⁸ represents hydrogen, methyl, propyl, propan-2-yl, 2-methoxyethan-1-yl, prop-2-en-1-yl, CH₂OCH₃, COMe, COOMe, COOEt, COOtertBu, COCF₃ or benzyl and
R⁹ represents cyclobutyl, 2-methylcycloprop-1-yl, 2-methylcyclobut-1-yl, 3-methylcyclobut-1-yl, 2,2-difluoroethyl, isopropyl, cyclopropylmethyl, 2,2-dimethylcyclopropyl, cyclopentyl, butan-2-yl, 1-methoxypropan-2-yl, 2-methyl-1-(methylsulphanyl)propan-2-yl, 1,1,1-trifluoropropan-2-yl or 2,2,3,3,3-pentafluoropropyl.

The substituted aminothiophenes (IV), hereinbelow also described as IVa or IVb,

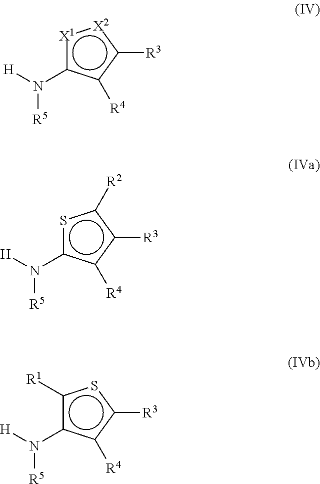

are either commercially available or can be prepared from commercially available precursors by methods known from the literature. Aminothiophenes carrying one or more identical or different substituents in the thiophene moiety can be prepared by a large number of methods described in the relevant literature. Some of the methods are mentioned hereinbelow by way of example:
f) the reduction of nitrothiophenes of the formula (X¹) in the presence of a reducing agent and in the presence of a solvent to give the aminothiophenes of the formula (IV-1), according to the reaction scheme below, where the radicals X¹, X² and R³ and R⁴ are defined analogously to those of formula (IV) (Scheme 6):

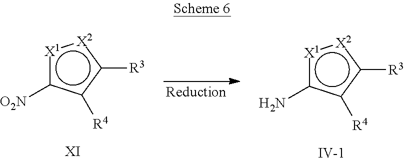

g) the reaction of thiophenecarboxylic acids of the formula (IV-2) in the presence of an organic azide (XII) and an auxiliary base and in the presence of a solvent to give thiophenecarbamates (IV-3), according to the reaction scheme below, where the radicals X¹, X² and R³ and R⁴ are defined analogously to those of formula (IV) (Scheme 7):

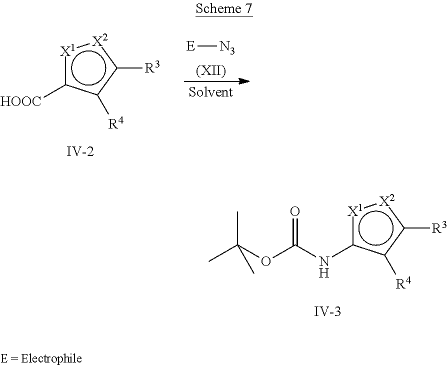

h) the reaction of thiophenecarbamates of the formula (IV-3) with the intermediates of the formula (V), if appropriate in the presence of an acid and in the presence of a solvent, to give the thienylaminopyrimidines of the formula (I), according to the reaction scheme below, where the radicals X¹, X² and R³ and R⁴ are defined analogously to those of formula (IV) (Scheme 8):

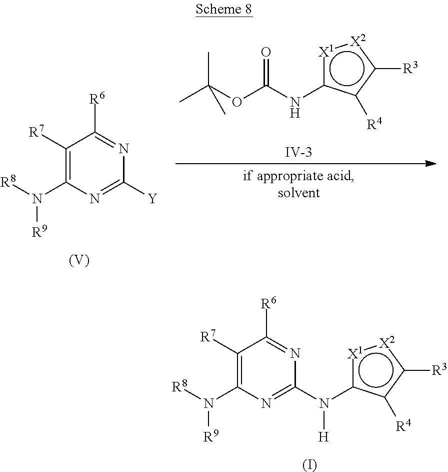

i) the reaction of thienylaminopyrimidines of the formula Ia in the presence of a base and in the presence of a solvent to give the thienylaminopyrimidines of the formula Ib (Scheme 9), where the radicals X¹, X² and R⁴ are defined analogously to those of formula (IV). One way of preparing compounds of the formula (Ia) is shown in Scheme 2.

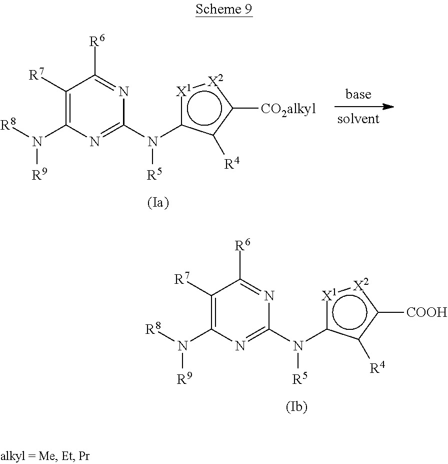

j) the reaction of thienylaminopyrimidines of the formula (Ib) in the presence of a coupling agent and an auxiliary base and in the presence of a solvent to give thienylaminopyrimidines of the formula (Ic), where the radicals X¹, X² and R⁴ are defined analogously to those of formula (IV) (Scheme 10).

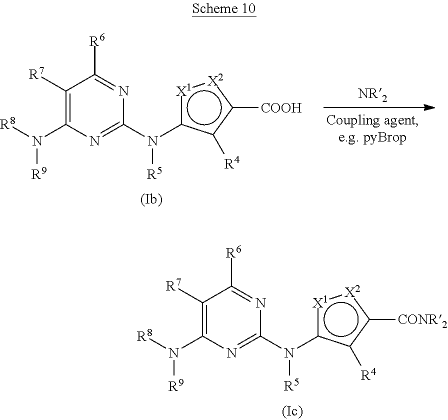

One way of preparing compounds of the formula (IV-1) is shown in Scheme 6.

Substituted aminothiophenes (IV-1) can be prepared, for example, by reacting commercially available and appropriately substituted nitrothiophenes (XI) with reducing agents (for example iron powder, zinc powder, tin powder, see, for example, Heterocycles 2005, 65, 2369-2380) in suitable solvents, such as, for example, acetic acid or hydrochloric acid, at temperatures of from 20° C. to 150° C., but preferably from 70° C. to 90° C. (Scheme 6).

One way of preparing compounds of the formula (IV-3) is shown in Scheme 7.

One method of synthesizing thiophenecarbamates of the formula (IV-3) consists in reacting commercially available thiophenecarboxylic acids (IV-2, Scheme 7) with organic azides (XII), such as, for example, phosphoryl azide, in the presence of a suitable base (for example triethylamine, diisopropylamine), using suitable solvents (for example tert-butanol, benzyl alcohol), to give the corresponding thiophenecarbamates (IV-3) (see, for example, WO 2007/076423; Bioorg. Med. Chem. Lett. 2006, 16, 5567-5571).

One way of preparing compounds of the formula (I) from compounds of the formula (IV-3) is shown in Scheme 8.

Here, the intermediate (V) is reacted in the presence of Brönsted acids, such as, for example, anhydrous hydrochloric acid, camphorsulphonic acid or p-toluenesulphonic acid, in a suitable solvent, such as, for example, dioxane, THF, DMSO, DME, 2-methoxyethanol, n-butanol or acetonitrile, at a temperature of 0° C.-140° C. over a period of 1-48 h with a thiophenecarbamate (IV-3).

One way of preparing compounds of the formula (Ib) is shown in Scheme 9.

Thus, for example, alkoxycarbonyl-substituted thienylaminopyrimidines of type (Ia) obtainable by the process shown in Scheme 2 can be converted, if appropriate in the presence of an acid and in the presence of a solvent, and are then hydrolyzed by reaction with suitable bases, such as, for example, NaOH or KOH, in suitable solvents, such as, for example, water, to give the corresponding carboxylic acid-substituted thienylaminopyrimidines (Ib) (see, for example, J. Med. Chem. 1986, 29, 1637-1643).

One way of preparing compounds of the formula (Ic) is shown in Scheme 10.

By reacting the carboxylic acid-substituted thienylaminopyrimidines (Ib) with amines using generally known coupling agents (for example bromotrispyrrolidinophosphonium hexafluorophosphate, PyBrop) in the presence of an auxiliary base and in the presence of a solvent, it is possible to generate the corresponding carboxamide-substituted thienylaminopyrimidines (Ic) (see, for example, WO 06/040569).

One way of preparing compounds of the formula (IX) is shown in Scheme 3.

2-Halo-substituted pyrimidin-4-ones (VI) are available by regioselective hydrolysis from 2,4-dihalo-substituted pyrimidines. This is described, for example, in Russ. J. Org. Chem. 2006, 42, 580; J. Med. Chem. 1965, 8, 253.

Intermediates of the formula (VI) are reacted in the presence of Brönsted acids, such as, for example, anhydrous hydrochloric acid, camphorsulphonic acid or p-toluenesulphonic acid, in a suitable solvent, such as, for example, dioxane, THF, DMSO, DME, 2-methoxyethanol, n-butanol or acetonitrile, at a temperature of 0° C.-140° C. over a period of 1-48 h with a thiophenamine (IV).

Alternatively, the reaction of (VI) and (IV) to give (IX) can also be carried out with base catalysis, i.e. using, for example, carbonates, such as potassium carbonate, alkoxides, such as potassium tert-butoxide, or hydrides, such as sodium hydride, where the catalytic use of a transition metal, such as, for example, palladium, together with a suitable ligand, such as, for example, xanthphos, may also be useful.

Finally, it is possible to carry out the reaction of (VI) and (IV) to give (IX) in the absence of solvents and/or Brönsted acids (described, for example, in Bioorg. Med. Chem. Lett. 2006, 16, 108; Bioorg. Med. Chem. Lett. 2005, 15, 3881).

Some of the compounds of the formula (IX) are novel, and thus also part of the subject-matter of the present invention.

Novel are compounds of the formula (IX)

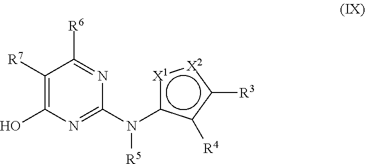

in which the symbols have the meanings below:
X¹, X², R¹ to R⁶, R⁷ have the general, preferred, particularly preferred, very particularly preferred and especially preferred meanings given above.

One way of preparing compounds of the formula (X) is shown in Scheme 4.

Intermediates of the formula (IX) can be converted by reaction with suitable halogenating agents, such as, for example, thionyl chloride, phosphorus pentoxide or phosphoryl chloride, or a mixture thereof, if appropriate in the presence of a suitable solvent, such as, for example, toluene or ethanol, and if appropriate in the presence of a suitable base, such as, for example, triethylamine, into 2-thiophenamino-4-chloropyrimidines of the formula (X). In an analogous manner, this is described, for example, in J. Med. Chem. 1989, 32, 1667; J. Heterocycl. Chem. 1989, 26, 313.

Some of the compounds of the formula (X) are novel and thus also part of the subject-matter of the present invention.

Novel are compounds of the formula (X)

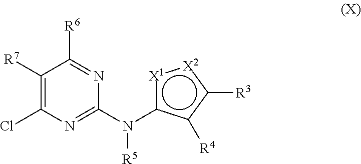

in which the symbols have the meanings given below:

X¹, X², R¹ to R⁷ have the general, preferred, particularly preferred, very particularly preferred and especially preferred meanings given above.

A further way preparing the compound (I) is shown in Scheme 5.

For preparing compounds of the formula (I), the intermediate (X) is reacted in the presence of bases, such as, for example, carbonates, such as potassium carbonate, alkoxides, such as potassium tert-butoxide, or hydrides, such as sodium hydride, in a suitable solvent, such as, for example, dioxane, THF, DMSO, DME, 2-methoxyethanol, n-butanol or acetonitrile, at a temperature of 0° C.-140° C. over a period of 1-48 h with amines of the formula (II), where the catalytic use of a transition metal, such as, for example, palladium, together with a suitable ligand, such as, for example, triphenylphosphine or xanthphos, may also be useful.

The processes according to the invention for preparing the compounds of the formula (I) are preferably carried out using one or more reaction auxiliaries.

Suitable reaction auxiliaries are, if appropriate, the customary inorganic or organic bases or acid acceptors. These preferably include alkali metal and alkaline earth metal acetates, amides, carbonates, bicarbonates hydrides, hydroxides, and alkoxides, such as, for example, sodium acetate, potassium acetate or calcium acetate, lithium amide, sodium amide, potassium amide or calcium amide, sodium carbonate, potassium carbonate or calcium carbonate, sodium bicarbonate, potassium bicarbonate or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n- or i-propoxide, n-, i-, s- or t-butoxide or potassium methoxide, ethoxide, n- or i-propoxide, n-, i-, s- or t-butoxide; furthermore also basic organic nitrogen compounds, such as, for example, trimethylamine, triethylamine, tripropylamine, tributylamine, ethyldiisopropylamine, N,N-dimethylcyclohexylamine, dicyclohexylamine, ethyldicyclohexylamine, N,N-dimethylaniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, 1,4-diazabicyclo[2.2.2]-octane (DABCO), 1,5-diazabicyclo[4.3.0]non-5-ene (DBN) or 1,8-diazabicyclo[5.4.0]undec-7-ene (DBU).

The processes according to the invention are preferably carried out using one or more diluents. Suitable diluents are virtually all inert organic solvents. These preferably include aliphatic and aromatic, optionally halogenated hydrocarbons, such as pentane, hexane, heptane, cyclohexane, petroleum ether, benzine, ligroin, benzene, toluene, xylene, methylene chloride, ethylene chloride, chloroform, carbon tetrachloride, chlorobenzene and o-dichlorobenzene, ethers, such as diethyl ether and dibutyl ether, glycol dimethyl ether and diglycol dimethyl ether, tetrahydrofuran and dioxane, ketones, such as acetone, methyl ethyl ketone, methyl isopropyl ketone or methyl isobutyl ketone, esters, such as methyl acetate or ethyl acetate, nitriles, such as, for example, acetonitrile or propionitrile, amides, such as, for example, dimethylformamide, dimethylacetamide and N-methylpyrrolidone, and also dimethyl sulphoxide, tetramethylene sulphone and hexamethyl-phosphoric triamide and DMPU.

In the processes according to the invention, the reaction temperatures can be varied within a relatively wide range. In general, the processes are carried out at temperatures between 0° C. and 250° C., preferably at temperatures between 10° C. and 185° C.

The processes according to the invention are generally carried out under atmospheric pressure. However, it is also possible to operate under elevated or reduced pressure.

For carrying out the processes according to the invention, the starting materials required in each case are generally employed in approximately equimolar amounts. However, it is also possible to use in each case one of the components employed in a relatively large excess. Work-up in the processes according to the invention is in each case carried out by customary methods (cf. the Preparation Examples).

In general, compounds of the formula (I) can be prepared, for example, by sequential nucleophilic addition of an aliphatic amine (II) and a heteroaromatic amine (IV) to a suitable substituted pyrimidine (III), as outlined below in Scheme 9:

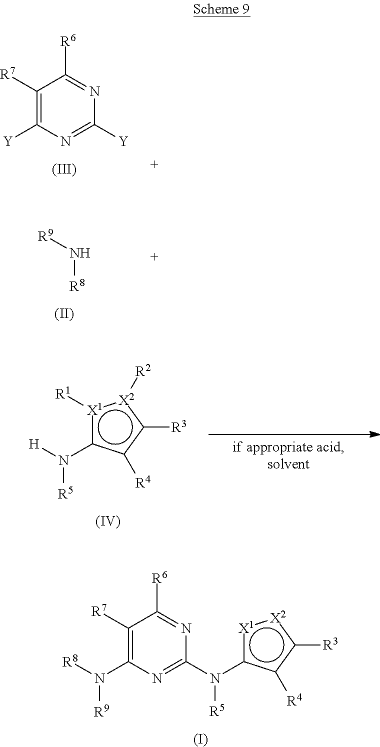

Here, Y, in each case independently of one another, represent suitable leaving groups, for example a halogen atom (Hal=F, Cl, Br, I), SMe, SO₂Me, SOMe or else triflate (CF₃SO₂O: for pyrimidines known from WO 2005/095386).

The synthesis of thienylaminopyrimidines of the formula (I) according to Scheme 8 or else by other routes has been described in the literature in many different instances (see, for example, also WO 2003/076437).

The invention furthermore provides the non-medicinal use of the thienylaminopyrimidines according to the invention or of mixtures of these for controlling unwanted microorganisms.

The invention furthermore provides a composition for controlling unwanted microorganisms, comprising at least one thienylaminopyrimidine according to the present invention.

Moreover, the invention relates to a method for controlling unwanted microorganisms, characterized in that the thienylaminopyrimidines according to the invention are applied to the microorganisms and/or their habitat.

The compounds according to the invention have strong microbicidal action and can be used for controlling unwanted microorganisms, such as fungi and bacteria, in crop protection and in the protection of materials.

The thienylaminopyrimidines of the formula (I) according to the invention have very good fungicidal properties and can be used in crop protection, for example for controlling Plasmodiophoromycetes, Oomycetes, Chytridiomycetes, Zygomycetes, Ascomycetes, Basidiomycetes and Deuteromycetes.

In crop protection, bactericides can be used for controlling Pseudomonadaceae, Rhizobiaceae, Enterobacteriaceae, Corynebacteriaceae and Streptomycetaceae.

The fungicidal compositions according to the invention can be used for the curative or protective control of phytopathogenic fungi. Accordingly, the invention also relates to curative and protective methods for controlling phytopathogenic fungi using the active compounds or compositions according to the invention, which are applied to the seed, the plant or plant parts, the fruit or the soil on which the plants grow.

The compositions according to the invention for controlling phytopathogenic fungi in crop protection comprise an effective, but non-phytotoxic amount of the active compounds according to the invention. “Effective, but non-phytotoxic amount” means an amount of the composition according to the invention which is sufficient to control the fungal disease of the plant in a satisfactory manner or to eradicate the fungal disease completely, and which, at the same time, does not cause any significant symptoms of phytotoxicity. In general, this application rate may vary within a relatively wide range. It depends on a plurality of factors, for example on the fungus to be controlled, the plant, the climatic conditions and the ingredients of the compositions according to the invention.

According to the invention, it is possible to treat all plants and parts of plants. Plants are to be understood here as meaning all plants and plant populations, such as wanted and unwanted wild plants or crop plants (including naturally occurring crop plants). Crop plants can be plants which can be obtained by conventional breeding and optimization methods or by biotechnological and genetic engineering methods or combinations of these methods, including the transgenic plants and including plant cultivars which can or cannot be protected by varietal property rights. Parts of plants are to be understood as meaning all above-ground and below-ground parts and organs of the plants, such as shoot, leaf, flower and root, examples which may be mentioned being leaves, needles, stems, trunks, flowers, fruit bodies, fruits and seeds and also roots, tubers and rhizomes. Plant parts also include harvested material and vegetative and generative propagation material, for example seedlings, tubers, rhizomes, cuttings and seeds.

The following plants may be mentioned as plants which can be treated according to the invention: cotton, flax, grapevines, fruit, vegetables, such as Rosaceae sp. (for example pomaceous fruit, such as apples and pears, but also stone fruit, such as apricots, cherries, almonds and peaches and soft fruit such as strawberries), Ribesioidae sp., Juglandaceae sp., Betulaceae sp., Anacardiaceae sp., Fagaceae sp., Moraceae sp., Oleaceae sp., Actimidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit), Solanaceae sp. (for example tomatoes), Liliaceae sp., Asteraceae sp. (for example lettuce), Umbelliferae sp., Cruciferae sp., Chenopodiaceae sp., Cucurbitaceae sp. (for example cucumbers), Alliaceae sp. (for example leek, onions), Papilionaceae sp. (for example peas); major crop plants, such Gramineae sp. (for example maize, lawn, cereals such as wheat, rye, rice, barley, oats, millet and triticale), Asteraceae sp. (for example sunflowers), Brassicaceae sp. (for example white cabbage, red cabbage, broccoli, cauliflowers, brussels sprouts, pak choi, kohlrabi, garden radish, and also oilseed rape, mustard, horseradish and cress), Fabacae sp. (for example beans, peanuts), Papilionaceae sp. (for example soya beans), Solanaceae sp. (for example potatoes), Chenopodiaceae sp. (for example sugarbeet, fodderbeet, swiss chard, beetroot); crop plants and ornamental plants in garden and forest; and also in each case genetically modified varieties of these plants. Preferably, cereal plants are treated according to the invention.

Some pathogens of fungal diseases which can be treated according to the invention may be mentioned by way of example, but not by way of limitation:

Diseases caused by powdery mildew pathogens, such as, for example, Blumeria species, such as, for example, Blumeria graminis; Podosphaera species, such as, for example, Podosphaera leuco-tricha; Sphaerotheca species, such as, for example, Sphaerotheca fuliginea; Uncinula species, such as, for example, Uncinula necator;

Diseases caused by rust disease pathogens, such as, for example, Gymnosporangium species, such as, for example, Gymnosporangium sabinae; Hemileia species, such as, for example, Hemileia vastatrix; Phakopsora species, such as, for example, Phakopsora pachyrhizi and Phakopsora meibomiae; Puccinia species, such as, for example, Puccinia recondita or Puccinia triticina; Uromyces species, such as, for example, Uromyces appendiculatus;

Diseases caused by pathogens from the group of the Oomycetes, such as, for example, Brenda species, such as, for example, Brenda lactucae; Peronospora species, such as, for example, Peronospora pisi or P. brassicae; Phytophthora species, such as, for example Phytophthora infestans; Plasmopara species, such as, for example, Plasmopara viticola; Pseudoperonospora species, such as, for example, Pseudoperonospora humuli or Pseudoperonospora cubensis; Pythium species, such as, for example, Pythium ultimum;

Leaf blotch diseases and leaf wilt diseases caused, for example, by Alternaria species, such as, for example, Alternaria solani; Cercospora species, such as, for example, Cercospora beticola; Cladiosporium species, such as, for example, Cladiosporium cucumerinum; Cochliobolus species, such as, for example, Cochliobolus sativus (conidia form: Drechslera, Syn: Helminthosporium); Colletotrichum species, such as, for example, Colletotrichum lindemuthanium; Cycloconium species, such as, for example, Cycloconium oleaginum; Diaporthe species, such as, for example, Diaporthe citri; Elsinoe species, such as, for example, Elsinoe fawcettii; Gloeosporium species, such as, for example, Gloeosporium laeticolor; Glomerella species, such as, for example, Glomerella cingulata; Guignardia species, such as, for example, Guignardia bidwelli; Leptosphaeria species, such as, for example, Leptosphaeria maculans; Magnaporthe species, such as, for example, Magnaporthe grisea; Microdochium species, such as, for example, Microdochium nivale; Mycosphaerella species, such as, for example, Mycosphaerella graminicola and M. fijiensis; Phaeosphaeria species, such as, for example, Phaeosphaeria nodorum; Pyrenophora species, such as, for example, Pyrenophora teres; Ramularia species, such as, for example, Ramularia collo-cygni; Rhynchosporium species, such as, for example, Rhynchosporium secalis; Septoria species, such as, for example, Septoria apii; Typhula species, such as, for example, Typhula incarnata; Venturia species, such as, for example, Venturia inaequalis;

Root and stem diseases caused, for example, by Corticium species, such as, for example, Corticium graminearum; Fusarium species, such as, for example, Fusarium oxysporum; Gaeumannomyces species, such as, for example, Gaeumannomyces graminis; Rhizoctonia species, such as, for example Rhizoctonia solani; Tapesia species, such as, for example, Tapesia acuformis; Thielaviopsis species, such as, for example, Thielaviopsis basicola;

Ear and panicle diseases (including maize cobs) caused, for example, by Alternaria species, such as, for example, Alternaria spp.; Aspergillus species, such as, for example, Aspergillus flavus; Cladosporium species, such as, for example, Cladosporium cladosporioides; Claviceps species, such as, for example, Claviceps purpurea; Fusarium species, such as, for example, Fusarium culmorum; Gibberella species, such as, for example, Gibberella zeae; Monographella species, such as, for example, Monographella nivalis; Septoria species, such as for example, Septoria nodorum;

Diseases caused by smut fungi, such as, for example, Sphacelotheca species, such as, for example, Sphacelotheca reiliana; Tilletia species, such as, for example, Tilletia caries; T. controversa; Urocystis species, such as, for example, Urocystis occulta; Ustilago species, such as, for example, Ustilago nuda; U. nuda tritici;

Fruit rot caused, for example, by Aspergillus species, such as, for example, Aspergillus flavus; Botrytis species, such as, for example, Botrytis cinerea; Penicillium species, such as, for example, Penicillium expansum and P. purpurogenum; Sclerotinia species, such as, for example, Sclerotinia sclerotiorum;

Verticilium species, such as, for example, Verticilium alboatrum;

Seed- and soil-borne rot and wilt diseases, and also diseases of seedlings, caused, for example, by Fusarium species, such as, for example, Fusarium culmorum; Phytophthora species, such as, for example, Phytophthora cactorum; Pythium species, such as, for example, Pythium ultimum; Rhizoctonia species, such as, for example, Rhizoctonia solani; Sclerotium species, such as, for example, Sclerotium rolfsii;

Cancerous diseases, galls and witches' broom caused, for example, by Nectria species, such as, for example, Nectria galligena;

Wilt diseases caused, for example, by Monilinia species, such as, for example, Monilinia laxa;

Deformations of leaves, flowers and fruits caused, for example, by Taphrina species, such as, for example, Taphrina deformans;

Degenerative diseases of woody plants caused, for example, by Esca species, such as, for example, Phaemoniella clamydospora and Phaeoacremonium aleophilum and Fomitiporia mediterranea;

Diseases of flowers and seeds caused, for example, by Botrytis species, such as, for example, Botrytis cinerea;

Diseases of plant tubers caused, for example, by Rhizoctonia species, such as, for example, Rhizoctonia solani; Helminthosporium species, such as, for example, Helminthosporium solani;

Diseases caused by bacteriopathogens, such as, for example, Xanthomonas species, such as, for example, Xanthomonas campestris pv. oryzae; Pseudomonas species, such as, for example, Pseudomonas syringae pv. lachrymans; Erwinia species, such as, for example, Erwinia amylovora.

Preference is given to controlling the following diseases of soya beans:

fungal diseases on leaves, stems, pods and seeds caused, for example, by alternaria leaf spot (Alternaria spec. atrans tenuissima), anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi Phakopsora meibomiae), scab (Sphaceloma glycines), stemphylium leaf blight (Stemphylium botryosum), target spot (Corynespora cassiicola).

Fungal diseases on roots and the stem base caused, for example, by black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmopspora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia Southern blight (Sclerotinia rolfsii), thielaviopsis root rot (Thielaviopsis basicola).

In the present case, undesired microorganisms are understood as meaning phytopathogenic fungi and bacteria. Thus, the compounds according to the invention can be employed for protecting plants against attack by the abovementioned pathogens within a certain period of time after the treatment. The period of time within which their protection is effected is generally extended from 1 to 10 days, preferably 1 to 7 days, after the plants have been treated with the active compounds.

The fact that the active compounds, at the concentrations required for the controlling of plant diseases, are well tolerated by plants permits the treatment of above-ground plant parts, of vegetative propagation material and seed, and of the soil.

In this context, the active compounds according to the invention can be employed particularly successfully for controlling cereal diseases such as, for example, against Erysiphe species, against Puccinia and against Fusaria species, rice diseases such as, for example against Pyricularia and Rhizoctonia and diseases in viticulture, fruit production and vegetable production such as, for example, against Botrytis, Venturia, Sphaerotheca and Podosphaera species.

The active compounds according to the invention are also suitable for increasing the yield. Moreover, they display a low degree of toxicity and are well tolerated by plants.

If appropriate, the compounds according to the invention can, at certain concentrations or application rates, also be used as herbicides, safeners, growth regulators or agents to improve plant properties, or as microbicides, for example as fungicides, antimycotics, bactericides, viricides (including agents against viroids) or as agents against MLO (Mycoplasma-like organisms) and RLO (Rickettsia-like organisms). If appropriate, they can also be employed as insecticides. If appropriate, they can also be employed as intermediates or precursors for the synthesis of other active compounds.

If appropriate, the active compounds according to the invention can also be used in certain concentrations and application rates as herbicides and for influencing plant growth, and also for controlling animal pests. If appropriate, they can also be employed as intermediates and precursors for the synthesis of further active compounds.

The active compounds according to the invention, in combination with good plant tolerance and favourable toxicity to warm-blooded animals and being tolerated well by the environment, are suitable for protecting plants and plant organisms, for increasing harvest yields and for improving the quality of harvested material. They are preferably employed as plant protection agents. They are active against normally sensitive and resistant species and against all or some stages of development.

The treatment according to the invention of the plants and plant parts with the active compounds or compositions is carried out directly or by action on their surroundings, habitat or storage space using customary treatment methods, for example by dipping, spraying, atomizing, irrigating, evaporating, dusting, fogging, broadcasting, foaming, painting, spreading-on, watering (drenching), drip irrigating and, in the case of propagation material, in particular in the case of seeds, furthermore as a powder for dry seed treatment, a solution for seed treatment, a water-soluble powder for slurry treatment, by encrusting, by coating with one or more coats, etc. It is furthermore possible to apply the active compounds by the ultra-low-volume method or to inject the active compound preparation or the active compound itself into the soil.

The application rate of active compound can vary within a substantial range. Essentially, it depends on the nature of the desired effect. In general, the application rates are between 1 g and 10 kg of active compound per hectare of soil area, preferably between 5 g and 5 kg per ha.

The advantageous effect of the compatibility with crop plants of the active compounds according to the invention is particularly pronounced at certain concentration ratios. However, the weight ratios of the active compounds in the active compound combinations can be varied within relatively wide ranges. In general, from 0.001 to 1000 parts by weight, preferably from 0.01 to 100 parts by weight, particularly preferably 0.05 to 20 parts by weight, of one of the compounds which improves crop plant compatibility (antidotes/safeners) mentioned above under (b′) are present per part by weight of active compound of the formula (I).

The active compounds according to the invention are generally applied in the form of finished formulations. However, the active compounds contained in the active compound combinations can, as individual formulations, also be mixed during use, i.e. be applied in the form of tank mixes.

In addition, by the treatment according to the invention it is possible to reduce the mycotoxin content in the harvested material and the foodstuffs and feedstuffs prepared therefrom. Particular, but not exclusive, mention may be made here of the following mycotoxins: deoxynivalenol (DON), nivalenol, 15-Ac-DON, 3-Ac-DON, T2- and HT2-toxin, fumonisins, zearalenone, moniliformin, fusarin, diacetoxyscirpenol (DAS), beauvericin, enniatin, fusaroproliferin, fusarenol, ochratoxins, patulin, ergot alkaloids and aflatoxins produced, for example, by the following fungi: Fusarium spec., such as Fusarium acuminatum, F. avenaceum, F. crookwellense, F. culmorum, F. graminearum (Gibberella zeae), F. equiseti, F. fujikoroi, F. musarum, F. oxysporum, F. proliferatum, F. poae, F. pseudograminearum, F. sambucinum, F. scirpi, F. semitectum, F. solani, F. sporotrichoides, F. langsethiae, F. subglutinans, F. tricinctum, F. verticillioides, inter alia, and also by Aspergillus spec., Penicillium spec., Claviceps purpurea, Stachybotrys spec., inter alia.

In the protection of materials, the compositions or active compounds according to the invention can furthermore be employed for protecting industrial materials against attack and destruction by unwanted microorganisms, such as, for example, fungi.

In the present context, industrial materials are understood as meaning nonlive materials which have been made for use in technology. For example, industrial materials which are to be protected by active compounds according to the invention from microbial modification or destruction can be glues, sizes, paper and board, textiles, leather, timber, paints and plastic articles, cooling lubricants and other materials which are capable of being attacked or destroyed by microorganisms. Parts of production plants, for example cooling-water circuits, which can be adversely affected by the multiplication of microorganisms may also be mentioned within the materials to be protected. Industrial materials which may be mentioned with preference for the purposes of the present invention are glues, sizes, paper and board, leather, timber, paints, cooling lubricants and heat-transfer fluids, especially preferably wood. The compositions or active compounds according to the invention can prevent disadvantageous effects such as rotting, decay, discoloration, decoloration or the formation of mould.

The method according to the invention for controlling unwanted fungi can also be employed for protecting storage goods. Here, storage goods are to be understood as meaning natural substances of vegetable or animal origin or process products thereof of natural origin, for which long-term protection is desired. Storage goods of vegetable origin, such as, for example, plants or plant parts, such as stems, leaves, tubers, seeds, fruits, grains, can be protected freshly harvested or after processing by (pre)drying, moistening, comminuting, grinding, pressing or roasting. Storage goods also include timber, both unprocessed, such as construction timber, electricity poles and barriers, or in the form of finished products, such as furniture. Storage goods of animal origin are, for example, hides, leather, furs and hairs. The active compounds according to the invention can prevent disadvantageous effects, such as rotting, decay, discoloration, decoloration or the formation of mould.

Microorganisms capable of degrading or changing the industrial materials which may be mentioned are, for example, bacteria, fungi, yeasts, algae and slime organisms. The active compounds according to the invention preferably act against fungi, in particular moulds, wood-discoloring and wood-destroying fungi (Basidiomycetes) and against slime organisms and algae. Microorganisms of the following genera may be mentioned as examples: Alternaria, such as Alternaria tenuis; Aspergillus, such as Aspergillus niger; Chaetomium, such as Chaetomium globosum; Coniophora, such as Coniophora puetana; Lentinus, such as Lentinus tigrinus; Penicillium, such as Penicillium glaucum; Polyporus, such as Polyporus versicolor; Aureobasidium, such as Aureobasidium pullulans; Sclerophoma, such as Sclerophoma pityophila; Trichoderma, such as Trichoderma viride; Escherichia, such as Escherichia coli; Pseudomonas, such as Pseudomonas aeruginosa; Staphylococcus, such as Staphylococcus aureus.

The present invention furthermore relates to a composition for controlling unwanted microorganisms comprising at least one of the thienylaminopyrimidines according to the invention. These are preferably fungicidal compositions comprising auxiliaries, solvents, carriers, surfactants or extenders suitable for use in agriculture.

According to the invention, a carrier is a natural or synthetic, organic or inorganic substance with which the active compounds are mixed or bonded for better applicability, in particular for application to plants or parts of plants or seed. The carrier, which may be solid or liquid, is generally inert and should be suitable for use in agriculture.

Suitable solid carriers are: for example ammonium salts and ground natural minerals, such as kaolins, clays, talc, chalk, quartz, attapulgite, montmorillonite or diatomaceous earth, and ground synthetic minerals, such as finely divided silica, alumina and silicates; suitable solid carriers for granules are: for example crushed and fractionated natural rocks, such as calcite, marble, pumice, sepiolite and dolomite, and also synthetic granules of inorganic and organic meals, and granules of organic material, such as paper, sawdust, coconut shells, maize cobs and tobacco stalks; suitable emulsifiers and/or foam-formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates; suitable dispersants are nonionic and/or ionic substances, for example from the classes of the alcohol/POE and/or POP ethers, acid and/or POP/POE esters, alkylaryl and/or POP/POE ethers, fat and/or POP/POE adducts, POE and/or POP polyol derivatives, POE and/or POP/sorbitan or sugar adducts, alkyl or aryl sulphates, sulphonates and phosphates, or the corresponding PO ether adducts. Furthermore suitable oligo- or polymers, for example those derived from vinylic monomers, from acrylic acid, from EO and/or PO alone or in combination with, for example, (poly)alcohols or (poly)amines. It is also possible to employ lignin and its sulphonic acid derivatives, unmodified and modified celluloses, aromatic and/or aliphatic sulphonic acids and their adducts with formaldehyde.

The active compounds can be converted to the customary formulations, such as solutions, emulsions, wettable powders, water- and oil-based suspensions, powders, dusts, pastes, soluble powders, soluble granules, granules for broadcasting, suspension-emulsion concentrates, natural materials impregnated with active compound, synthetic materials impregnated with active compound, fertilizers and also microencapsulations in polymeric substances.

The active compounds can be used as such, in the form of their formulations or the use forms prepared therefrom, such as ready-to-use solutions, emulsions, water- or oil-based suspensions, powders, wettable powders, pastes, soluble powders, dusts, soluble granules, granules for broadcasting, suspension-emulsion concentrates, natural materials impregnated with active compound, synthetic materials impregnated with active compound, fertilizers and also microencapsulations in polymeric substances. Application is carried out in a customary manner, for example by pouring, spraying, atomizing, broadcasting, dusting, foaming, painting-on, etc. It is furthermore possible to apply the active compounds by the ultra-low-volume method or to inject the preparation of active compound or the active compound itself into the soil. It is also possible to treat the seed of the plants.

The formulations mentioned can be prepared in a manner known per se, for example by mixing the active compounds with at least one customary extender, solvent or diluent, emulsifier, dispersant and/or binder or fixative, wetting agent, water repellant, if appropriate siccatives and UV stabilizers and if appropriate colorants and pigments, antifoams, preservatives, secondary thickeners, glues, gibberellins and other processing auxiliaries.

The compositions according to the invention include not only formulations which are already ready to use and can be applied to the plant or the seed using a suitable apparatus, but also commercial concentrates which have to be diluted with water prior to use.

The active compounds according to the invention can be present as such or in their (commercial) formulations and also in the use forms prepared from these formulations as a mixture with other (known) active compounds, such as insecticides, attractants, sterilants, bactericides, acaricides, nematicides, fungicides, growth regulators, herbicides, fertilizers, safeners and/or semiochemicals.

Suitable for use as auxiliaries are substances which are suitable for imparting to the composition itself and/or to preparations derived therefrom (for example spray liquors, seed dressings) particular properties such as certain technical properties and/or also particular biological properties. Typical suitable auxiliaries are: extenders, solvents and carriers.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

Liquefied gaseous extenders or carriers are liquids which are gaseous at ambient temperature and under atmospheric pressure, for example aerosol propellants, such as halogenated hydrocarbons, and also butane, propane, nitrogen and carbon dioxide.

Tackifiers, such as carboxymethylcellulose and natural and synthetic polymers in the form of powders, granules and latices, such as gum arabic, polyvinyl alcohol, polyvinyl acetate, or else natural phospholipids, such as cephalins and lecithins and synthetic phospholipids can be used in the formulations. Other possible additives are mineral and vegetable oils.

If the extender used is water, it is also possible to use, for example, organic solvents as auxiliary solvents. Suitable liquid solvents are essentially: aromatic compounds, such as xylene, toluene or alkylnaphthalenes, chlorinated aromatic compounds or chlorinated aliphatic hydrocarbons, such as chlorobenzenes, chloroethylenes or methylene chloride, aliphatic hydrocarbons, such as cyclohexane or paraffins, for example mineral oil fractions, alcohols, such as butanol or glycol, and also ethers and esters thereof, ketones, such as acetone, methyl ethyl ketone, methyl isobutyl ketone or cyclohexanone, strongly polar solvents, such as dimethylformamide and dimethyl sulphoxide, and also water.

The compositions according to the invention may additionally comprise further components, such as, for example, surfactants. Suitable surfactants are emulsifiers and/or foam-formers, dispersants or wetting agents having ionic or nonionic properties, or mixtures of these surfactants. Examples of these are salts of polyacrylic acid, salts of lignosulphonic acid, salts of phenolsulphonic acid or naphthalenesulphonic acid, polycondensates of ethylene oxide with fatty alcohols or with fatty acids or with fatty amines, substituted phenols (preferably alkylphenols or arylphenols), salts of sulphosuccinic esters, taurine derivatives (preferably alkyl taurates), phosphoric esters of polyethoxylated alcohols or phenols, fatty esters of polyols, and derivatives of the compounds containing sulphates, sulphonates and phosphates, for example alkylaryl polyglycol ethers, alkylsulphonates, alkyl sulphates, arylsulphonates, protein hydrolysates, lignosulphite waste liquors and methylcellulose. The presence of a surfactant is required if one of the active compounds and/or one of the inert carriers is insoluble in water and the application is carried out in water. The proportion of surfactants is between 5 and 40 percent by weight of the compositions according to the invention.

It is possible to use colorants such as inorganic pigments, for example iron oxide, titanium oxide, Prussian blue, and organic dyes, such as alizarin dyes, azo dyes and metal phthalocyanine dyes, and trace nutrients, such as salts of iron, manganese, boron, copper, cobalt, molybdenum and zinc.

Other possible additives are perfumes, mineral or vegetable oils, if appropriate modified, waxes and nutrients (including trace nutrients), such as salts of iron, manganese, boron, copper, cobalt, molybdenum and zinc.

Stabilizers, such as low-temperature stabilizers, preservatives, antioxidants, light stabilizers or other agents which improve chemical and/or physical stability may also be present.

If appropriate, it is also possible for other addition components to be present, for example protective colloids, binders, glues, thickeners, thixotropic agents, penetrants, stabilizers, sequestrants, complex fomers. In general, the active compounds can be combined with any solid or liquid additive customarily used for formulation purposes.

The formulations generally comprise between 0.05 and 99% by weight, 0.01 and 98% by weight, preferably between 0.1 and 95% by weight, particularly preferably between 0.5 and 90% by weight, of active compound, very particularly preferably between 10 and 70 percent by weight.

The formulations described above can be employed in a method according to the invention for controlling unwanted microorganisms where the thienylaminopyrimidines according to the invention are applied to the microorganisms and/or their habitat.

The active compounds according to the invention, as such or in their formulations, can also be used in a mixture with known fungicides, bactericides, acaricides, nematicides or insecticides, for example to broaden the activity spectrum or to prevent the development of resistance.

Suitable mixing partners are, for example, known fungicides, insecticides, acaricides, nematicides or else bactericides (see also Pesticide Manual, 13th ed.).

A mixture with other known active compounds, such as herbicides, or with fertilizers and growth regulators, safeners and/or semiochemicals is also possible.

Application is carried out in a manner adapted to the use forms.

The control of phytopathogenic harmful fungi is primarily by treating the soil and the above-ground parts of the plants with crop protection compositions. Owing to concerns with a view to a possible impact of the crop protection compositions on the environment and human and animal health, there are efforts to reduce the amount of active compounds applied.

The active compounds can be applied as such, in the form of their formulations and the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, painting-on, etc. It is also possible to apply the active compounds by the ultra-low-volume method or to inject the preparation of active compound or the active compound itself into the soil. It is also possible to treat the seed of the plants.

When using the active compounds according to the invention as fungicides, the application rates can be varied within a relatively wide range, depending on the type of application. The application rate of the active compounds according to the invention is
in the treatment of parts of plants, for example leaves: from 0.1 to 10 000 g/ha, preferably from 10 to 1000 g/ha, particularly preferably from 50 to 300 g/ha (when the application is by watering or dripping, it is possible to reduce the application rate even more, in particular when inert substrates such as rock wool or perlite are used);
in the treatment of seed: from 2 to 200 g per 100 kg of seed, preferably from 3 to 150 g per 100 kg of seed, particularly preferably from 2.5 to 25 g per 100 kg of seed, very particularly preferably from 2.5 to 12.5 g per 100 kg of seed;
in soil treatment: from 0.1 to 10 000 g/ha, preferably from 1 to 5000 g/ha.

These application rates are mentioned only in an exemplary manner and not limiting for the purpose of the invention.

The compounds according to the invention can also be used for protecting objects which come into contact with salt water or brackish water, such as hulls, screens, nets, buildings, moorings and signalling systems, against colonization.

The active compounds according to the invention, alone or in combination with other active compounds, can furthermore be employed as antifouling agents.

The treatment method according to the invention can be used for treating genetically modified organisms (GMOs), for example plants or seeds. Genetically modified plants (or transgenic plants) are plants in which a heterologous gene has been stably integrated into the genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example, antisense technology, cosuppression technology or RNA interference—RNAi—technology). A heterologous gene that is located in the genome is also called a transgene. A transgene that is defined by its particular location in the plant genome is called a transformation or transgenic event.

Depending on the plant species or plant cultivars, their location and growth conditions (soils, climate, vegetation period, diet), the treatment according to the invention may also result in superadditive (“synergistic”) effects. Thus, for example, the following effects, which exceed the effects which were actually to be expected, are possible: reduced application rates and/or a widening of the activity spectrum and/or an increase in the activity of the active compounds and compositions which can be used according to the invention, better plant growth, increased tolerance to high or low temperatures, increased tolerance to drought or to water or soil salt content, increased flowering performance, easier harvesting, accelerated maturation, higher harvest yields, bigger fruits, larger plant height, greener leaf colour, earlier flowering, higher quality and/or a higher nutritional value of the harvested products, higher sugar concentration within the fruits, better storage stability and/or processability of the harvested products.

In the present case, unwanted phytopathogenic fungi and/or microorganisms and/or viruses are to be understood as meaning phytopathogenic fungi, bacteria and viruses. Thus, the substances according to the invention can be employed for protecting plants against attack by the abovementioned pathogens within a certain period of time after the treatment. The period of time within which protection is effected generally extends from 1 to 10 days, preferably 1 to 7 days, after the treatment of the plants with the active compounds.

Plants and plant cultivars which are preferably treated according to the invention include all plants with genetic material which bestows upon these plants particularly advantageous useful properties (whether this was achieved by breeding and/or biotechnology is immaterial).

Plants and plant cultivars which are also preferably treated according to the invention are resistant towards one or more biotic stresses, i.e. said plants have a better defense against animal and microbial pests, such as against nematodes, insects, mites, phytopathogenic fungi, bacteria, viruses and/or viroids.

Plants and plant cultivars which may also be treated according to the invention are those which are resistant to one or more abiotic stress factors. Abiotic stress conditions may include, for example, drought, cold temperature exposure, heat exposure, osmotic stress, flooding, increased soil salinity, increased mineral exposure, ozone exposure, high light exposure, limited availability of nitrogen nutrients, limited availability of phosphorus nutrients or shade avoidance.

Plants and plant cultivars which may also be treated according to the invention, are those plants characterized by enhanced yield characteristics. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore by affected by improved plant architecture (under stress and non-stress conditions), including early flowering, flowering control for hybrid seed production, seedling vigour, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content, protein content, oil content and composition, nutritional value, reduction in anti-nutritional compounds, improved processability and better storage stability.

Plants that may be treated according to the invention are hybrid plants that already express the characteristics of heterosis or the hybrid effect which results in generally higher yield, vigour, health and resistance towards biotic and abiotic stress factors. Such plants are typically made by crossing an inbred male sterile parent line (the female parent) with another inbred male fertile parent line (the male parent). Hybrid seed is typically harvested from the male sterile plants and sold to growers. Male sterile plants can sometimes (e.g. in corn) be produced by detasseling, (i.e. the mechanical removal of the male reproductive organs or male flowers) but, more typically, male sterility is the result of genetic determinants in the plant genome. In that case, and especially when seed is the desired product to be harvested from the hybrid plants, it is typically useful to ensure that male fertility in the hybrid plants, which contain the genetic determinants responsible for male sterility, is fully restored. This can be accomplished by ensuring that the male parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described in Brassica species. However, genetic determinants for male sterility can also be located in the nuclear genome. Male sterile plants can also be obtained by plant biotechnology methods such as genetic engineering. A particularly useful means of obtaining male sterile plants is described in WO 89/10396 in which, for example, a ribonuclease such as a barnase is selectively expressed in the tapetum cells in the stamens. Fertility can then be restored by expression in the tapetum cells of a ribonuclease inhibitor such as barstar.

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-tolerant plants are for example glyphosate-tolerant plants, i.e. plants made tolerant to the herbicide glyphosate or salts thereof. For example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium, the CP4 gene of the bacterium Agrobacterium sp., the genes encoding a petunia EPSPS, a tomato EPSPS, or an Eleusine EPSPS. It can also be a mutated EPSPS. Glyphosate-tolerant plants can also be obtained by expressing a gene that encodes a glyphosate oxidoreductase enzyme. Glyphosate-tolerant plants can also be obtained by expressing a gene that encodes a glyphosate acetyl transferase enzyme. Glyphosate-tolerant plants can also be obtained by selecting plants containing naturally-occurring mutations of the above-mentioned genes.

Other herbicide-resistant plants are for example plants that are made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition. One such efficient detoxifying enzyme is, for example, an enzyme encoding a phosphinothricin acetyltransferase (such as the bar or pat protein from Streptomyces species). Plants expressing an exogenous phosphinothricin acetyltransferase have been described.

Further herbicide-tolerant plants are also plants that are made tolerant to the herbicides inhibiting the enzyme hydroxyphenylpyruvatedioxygenase (HPPD). Hydroxyphenylpyruvatedioxygenases are enzymes that catalyse the reaction in which para-hydroxyphenylpyruvate (HPP) is transformed into homogentisate. Plants tolerant to HPPD-inhibitors can be transformed with a gene encoding a naturally-occurring resistant HPPD enzyme, or a gene encoding a mutated HPPD enzyme. Tolerance to HPPD-inhibitors can also be obtained by transforming plants with genes encoding certain enzymes enabling the formation of homogentisate despite the inhibition of the native HPPD enzyme by the HPPD-inhibitor. Tolerance of plants to HPPD inhibitors can also be improved by transforming plants with a gene encoding an enzyme prephenate dehydrogenase in addition to a gene encoding an HPPD-tolerant enzyme.

Still further herbicide-resistant plants are plants that are made tolerant to acetolactate synthase (ALS) inhibitors. Known ALS-inhibitors include, for example, sulphonylurea, imidazolinone, triazolopyrimidines, pyrimidinyloxy(thio)benzoates, and/or sulphonylaminocarbonyltriazolinone herbicides. Different mutations in the ALS enzyme (also known as acetohydroxyacid synthase, AHAS) are known to confer tolerance to different herbicides and groups of herbicides. The production of sulphonylurea-tolerant plants and imidazolinone-tolerant plants has been described in the international publication WO 96/033270. Further sulphonylurea- and imidazolinone-tolerant plants have also been described, for example in WO 07/024,782.

Other plants tolerant to imidazolinone and/or sulphonylurea can be obtained by induced mutagenesis, by selection in cell cultures in the presence of the herbicide or by mutation breeding. Plants or plant cultivars (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

In the present context, the term “insect-resistant transgenic plant” includes any plant containing at least one transgene comprising a coding sequence encoding:
1) an insecticidal crystal protein from Bacillus thuringiensis or an insecticidal portion thereof, such as the insecticidal crystal proteins listed online at: http://www.lifesci.sussex.ac,uk/Home/Neil Crickmore/Bl, or insecticidal portions thereof, for example proteins of the Cry protein classes Cry1Ab, Cry1Ac, Cry1F, Cry2Ab, Cry3Ae or Cry3Bb or insecticidal portions thereof; or
2) a crystal protein from Bacillus thuringiensis or a portion thereof which is insecticidal in the presence of a second other crystal protein from Bacillus thuringiensis or a portion thereof, such as the binary toxin made up of the Cy34 and Cy35 crystal proteins; or
3) a hybrid insecticidal protein comprising parts of two different insecticidal crystal proteins from Bacillus thuringiensis, such as a hybrid of the proteins of 1) above or a hybrid of the proteins of 2) above, for example the Cry1A.105 protein produced by maize event MON98034 (WO 07/027,777); or
4) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation, such as the Cry3Bb1 protein in maize events MON863 or MON88017, or the Cry3A protein in maize event MIR604;
5) an insecticidal secreted protein from Bacillus thuringiensis or Bacillus cereus, or an insecticidal portion thereof, such as the vegetative insecticidal proteins (VIP) listed at: http://www.lifesci.sussex.ac.uk/home/Neil_Crickmore/Bt/vip.html, for example proteins from the VIP3Aa protein class; or
6) a secreted protein from Bacillus thuringiensis or Bacillus cereus which is insecticidal in the presence of a second secreted protein from Bacillus thuringiensis or B. cereus, such as the binary toxin made up of the VIP1A and VIP2A proteins;
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation (while still encoding an insecticidal protein), such as the VIP3Aa protein in cotton event COT102.

Of course, insect-resistant transgenic plants, as used herein, also include any plant comprising a combination of genes encoding the proteins of any one of the above classes 1 to 8. In one embodiment, an insect-resistant plant contains more than one transgene encoding a protein of any one of the above classes 1 to 8, to expand the range of target insect species affected or to delay insect resistance development to the plants, by using different proteins insecticidal to the same target insect species but having a different mode of action, such as binding to different receptor binding sites in the insect.

Plants or plant cultivars (obtained by plant biotechnology methods such as genetic engineering) which may also be treated according to the invention are tolerant to abiotic stress factors. Such plants can be obtained by genetic transformation, or by selection of plants containing a mutation imparting such stress resistance. Particularly useful stress tolerance plants include:
a. plants which contain a transgene capable of reducing the expression and/or the activity of the poly(ADP-ribose)polymerase (PARP) gene in the plant cells or plants.
b. plants which contain a stress tolerance-enhancing transgene capable of reducing the expression and/or the activity of the PARG encoding genes of the plants or plants cells;
c. plants which contain a stress tolerance-enhancing transgene coding for a plant-functional enzyme of the nicotinamide adenine dinucleotide salvage biosynthesis pathway, including nicotinamidase, nicotinate phosphoribosyltransferase, nicotinic acid mononucleotide adenyl transferase, nicotinamide adenine dinucleotide synthetase or nicotinamide phosphoribosyltransferase.

Plants or plant cultivars (obtained by plant biotechnology methods such as genetic engineering) which may also be treated according to the invention show altered quantity, quality and/or storage-stability of the harvested product and/or altered properties of specific ingredients of the harvested product such as, for example:
1) transgenic plants which synthesize a modified starch, which in its physical-chemical characteristics, in particular the amylose content or the amylose/amylopectin ratio, the degree of branching, the average chain length, the side chain distribution, the viscosity behaviour, the gelling strength, the starch grain size and/or the starch grain morphology, is changed in comparison with the synthesized starch in wild type plant cells or plants, so that this modified starch is better suited for special applications.
2) transgenic plants which synthesize non-starch carbohydrate polymers or which synthesize non-starch carbohydrate polymers with altered properties in comparison to wild type plants without genetic modification. Examples are plants which produce polyfructose, especially of the inulin and levan type, plants which produce alpha-1,4-glucans, plants which produce alpha-1,6 branched alpha-1,4-glucans, and plants producing alternan.
3) transgenic plants which produce hyaluronan.

Plants or plant cultivars (that can be obtained by plant biotechnology methods such as genetic engineering) which may also be treated according to the invention are plants, such as cotton plants, with altered fibre characteristics. Such plants can be obtained by genetic transformation, or by selection of plants containing a mutation imparting such altered fibre characteristics and include:
a) plants, such as cotton plants, which contain an altered form of cellulose synthase genes,
b) plants, such as cotton plants, which contain an altered form of rsw2 or rsw3 homologous nucleic acids;
c) plants, such as cotton plants, with an increased expression of sucrose phosphate synthase;
d) plants, such as cotton plants, with an increased expression of sucrose synthase;
e) plants, such as cotton plants, wherein the timing of the plasmodesmatal gating at the basis of the fibre cell is altered, for example through downregulation of fibre-selective β-1,3-glucanase;
f) plants, such as cotton plants, which have fibres with altered reactivity, for example through the expression of the N-acetylglucosaminetransferase gene including nodC and chitin synthase genes.

Plants or plant cultivars (that can be obtained by plant biotechnology methods such as genetic engineering) which may also be treated according to the invention are plants, such as oilseed rape or related Brassica plants, with altered oil profile characteristics. Such plants can be obtained by genetic transformation or by selection of plants containing a mutation imparting such altered oil characteristics and include:
a) plants, such as oilseed rape plants, which produce oil having a high oleic acid content;
b) plants, such as oilseed rape plants, which produce oil having a low linolenic acid content;
c) plants, such as oilseed rape plants, which produce oil having a low level of saturated fatty acids.

Particularly useful transgenic plants which may be treated according to the invention are plants which comprise one or more genes which encode one or more toxins, are the following which are sold under the trade names YIELD GARD® (for example maize, cotton, soya beans), KnockOut® (for example maize), BiteGard® (for example maize), Bt-Xtra® (for example maize), StarLink® (for example maize), Bollgard® (cotton), Nucotn® (cotton), Nucotn 33B® (cotton), NatureGard® (for example maize), Protecta® and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya beans), Liberty Link® (tolerance to phosphinothricin, for example oilseed rape), IMI® (tolerance to imidazolinone) and SCS® (tolerance to sulphonylurea, for example maize). Herbicide-resistant plants (plants bred in a conventional manner for herbicide tolerance) which may be mentioned include the varieties sold under the name Clearfield® (for example maize).

Particularly useful transgenic plants which may be treated according to the invention are plants containing transformation events, or a combination of transformation events, that are listed for example in the databases for various national or regional regulatory agencies (see for example http://gmoinfo.jrc.it/gmp_browse.aspx and http://www.agbios.com/dbase.php).

According to the invention, the plants listed can be treated particularly advantageously with the compounds of the general formula (I) according to the invention. The preferred ranges indicated above for the active compounds and mixtures also apply to the treatment of these plants. Particular emphasis is given to treating the plants with the compounds and mixtures specifically indicated in the present text.

The compositions or active compounds according to the invention can also be used to protect plants for a certain period after treatment against attack by the pathogens mentioned. The period for which protection is provided generally extends over 1 to 28 days, preferably over 1 to 14 days, particularly preferably over 1 to 10 days, very particularly preferably over 1 to 7 days, after the treatment of the plants with the active compounds, or over up to 200 days after seed treatment. Preparation and use of the active compounds of the formula (I) according to the invention is shown in the examples below. However, the invention is not limited to these examples.

### Preparation of Starting Materials of the Formula (V)

### 2,5-Dichloro-N-cyclobutylpyrimidin-4-amine (V-1)

At −10° C., 3.39 g (24.5 mmol) of potassium carbonate are added to a solution of 3.00 g (16.4 mmol) of 2,4,5-trichloropyrimidine in 50 ml of acetonitrile. 1.22 g (17.2 mmol) of cyclobutylamine are then added as a 20% strength solution in acetonitrile. With stirring, the reaction mixture is allowed to warm to room temperature overnight. The reaction mixture is stirred into 250 ml of ice-water/dilute hydrochloric acid (1:1). The mixture is extracted with ethyl acetate (2×200 ml), the combined organic phases are then washed with water (2×100 ml) and dried over MgSO₄ and the solvent is removed under reduced pressure. This gives 3.45 g (94%) of 2,5-dichloro-N-cyclobutylpyrimidin-4-amine (V-1) (logP (pH 2.3): 2.62).

The following compounds can be prepared in an analogous manner:

### 5-Bromo-2-chloro-N-cyclobutylpyrimidin-4-amine (V-2) (logP (pH 2.3): 2.87).

### 2-Chloro-N-cyclobutyl-5-iodopyrimidin-4-amine (V-3) (logP (pH 2.3): 3.08).

### 2-Chloro-N-cyclobutyl-5-trifluoromethylpyrimidin-4-amine (V-4)

A mixture of 8.07 g (37.2 mmol) of 2,4-dichloro-5-trifluoropyrimidine and 12.8 g (92.9 mmol) of potassium carbonate in 150 ml of acetonitrile is warmed to 50° C. 4.00 g (37.2 mmol) of cyclobutylamine hydrochloride are then added, and the mixture is stirred for 2 h. After cooling, the reaction mixture is stirred into 500 ml of ice-water and extracted with ethyl acetate (3×200 ml).

The combined organic phases are separated off, washed with water (2×250 ml), dried over MgSO₄ and freed from the solvent under reduced pressure. The crude product is purified by column chromatography on silica gel (cyclohexane/ethyl acetate). This gives 4.00 g (41%) of 2-chloro-N-cyclobutyl-5-trifluoromethylpyrimidin-4-amine (V-4) (logP (pH 2.3): 3.20).

### Preparation of Compounds of the Formula (Iv)

### 1-(5-Amino-2-thienyl)ethanone (IV-1, Scheme 6)

At room temperature, 4.20 g of iron powder (75.2 mmol) are added to a solution of 3.00 g (17.5 mmol) of 1-(5-nitro-2-thienyl)ethanone in 360 ml of acetic acid, and the mixture is heated at 75° C. for 30 min. After cooling to room temperature, the reaction mixture is concentrated and taken up in 100 ml of ethyl acetate and 200 ml of saturated sodium bicarbonate solution. The organic phase is separated off, and the aqueous phase is extracted two more times with ethyl acetate. The combined organic phases are dried over sodium sulphate and filtered, and the solvent is removed under reduced pressure. This gives 1.50 g (60%) of the desired product (logP (pH 2.3): 0.42); ¹H NMR (400 MHz, DMSO-d6) δ=7.42 (d, 1H), 6.67 (s, 2H), 5.91 (d, 1H), 2.25 (s, 3H).

### 2-(N-tert-Butoxycarbonylamino)-5-methylthiophene (IV-3, Scheme 7)

3.87 g (14.0 mmol) of diphenylphosphoryl azide and 1.42 g (14.0 mmol) of triethylamine are added to a solution of 2.00 g (14.0 mmol) of 5-methylthiophene-2-carboxylic acid in 20 ml of dry tert-butanol. The reaction mixture is heated at 85° C. for 14 h. After cooling to room temperature, 100 ml of water are added and the mixture is extracted with ethyl acetate. The combined organic phases are dried over sodium sulphate and filtered, and the solvent is removed under reduced pressure. The crude product is then purified by column chromatography (cyclohexane/ethyl acetate 4:1). This gives 2.10 g (70%) of the desired product (logP (pH 2.3): 2.89); ¹H NMR (400 MHz, DMSO-d6) δ=9.35 (s, 1H), 6.41-6.43 (m, 1H), 6.31 (d, 1H), 2.30 (s, 3H), 1.45 (s, 9H).

### Preparation of Compounds of the Formula (I)

### N⁴-Cyclopropyl-N²-2-acetylthiophen-5-yl-5-(chloro)pyrimidine-2,4-diamine (Example 10, Scheme 2)

A mixture of 863 mg (4.23 mmol) of 2-chloro-N-cyclopropyl-5-(chloro)pyrimidin-4-amine, 717 mg (5.07 mmol) of 1-(5-amino-2-thienyl)ethanone and 644 mg (3.38 mmol) of 4-toluenesulphonic acid in 50 ml of dioxane is stirred at 105° C. for 16 h. After cooling, the reaction mixture is stirred into ice-water and extracted with ethyl acetate (3×100 ml). The combined organic phases are dried over Na₂SO₄, filtered and freed from the solvent under reduced pressure. Purification by column chromatography (cyclohexane/ethyl acetate 1:1) gives 806 mg (62%) of the desired product (logP (pH 2.3): 1.87); ¹H NMR (400 MHz, DMSO-d6) δ=8.01 (s, 1H), 7.64 (d, 1H), 7.24 (s, 1H), 6.69 (d, 1H), 2.95-3.00 (m, 1H), 2.37 (s, 3H), 0.84-0.88 (m, 2H), 0.70-0.73 (m, 2H).

### Preparation of Compounds of the Formula (Ia)

### 5-Chloro-N⁴-cyclopropyl-N²-2-methoxycarbonylthiophen-4-yl-2,4-diamine (Example 5, Scheme 2)

A mixture of 250 mg (1.23 mmol) of 5-chloro-2-chloro-N-cyclopropylpyrimidin-4-amine, 231 mg (1.47 mmol) of methyl 4-aminothiophene-2-carboxylate and 186 mg (0.98 mmol) of 4-toluenesulphonic acid in 12 ml of dioxane is stirred at 105° C. for 18 h. After cooling, the reaction mixture is stirred into ice-water and extracted with ethyl acetate (3×100 ml). The combined organic phases are washed with 50 ml of saturated aq. NaHCO₃ and dried over Na₂SO₄, filtered and freed from the solvent under reduced pressure. This gives 200 mg (50%) of the desired product (logP (pH 2.3): 1.68); ¹H NMR (400 MHz, DMSO-d6) δ=9.56 (s, 1H), 8.07 (s, 1H), 7.92 (s, 1H), 7.86 (s, 1H), 7.06 (s, 1H), 3.80 (s, 3H), 2.83-2.86 (m, 1H), 0.79-0.84 (m, 2H), 0.63-0.67 (m, 2H).

### Preparation of Compounds of the Formula (Ib)

### 5-Chloro-N⁴-cyclopropyl-N²-2-hydroxycarbonylthiophen-4-yl-2,4-diamine (Example 37, Scheme 9)

345 mg (3.08 mmol) of potassium tert-butoxide are added to a mixture of 500 mg (1.54 mmol) of 5-chloro-N⁴-cyclopropyl-N²-2-methoxycarbonylthiophen-4-yl-2,4-diamine and 20 ml of water, and the reaction mixture is heated at 100° C. for 3 h. After cooling to room temperature, the mixture is acidified with dilute hydrochloric acid and the resulting solid is filtered off with suction and dried. This gives 300 mg (63%) of the desired product (logP (pH 2.3): 1.30); ¹H NMR (400 MHz, DMSO-d6) δ=7.97-7.98 (m, 2H), 7.83 (d, 1H), 7.52 (s, 1H), 2.85-2.87 (m, 1H), 0.82-0.86 (m, 2H), 0.67-0.71 (m, 2H).

### Preparation of Compounds of the Formula (Ic)

### 5-Chloro-N⁴-cyclopropyl-N²-2-pyrrolidin-1-ylcarbonylthiophen-4-yl-2,4-diamine (Example 6, Scheme 10)

A mixture of 150 mg (0.48 mmol) of 5-chloro-N⁴-cyclopropyl-N²-2-hydroxycarbonylthiophen-4-yl-2,4-diamine, 29 mg (0.41 mmol) of pyrrolidine, 281 mg of Pybrop (0.60 mmol) and 104 mg (0.81 mmol) of diisopropylethylamine in 10 ml of dichloromethane is stirred at room temperature for 24 h. The reaction mixture is then washed with water, dried over sodium sulphate, filtered and concentrated. The crude product is then purified by column chromatography (cyclohexane/ethyl acetate 1:1). This gives 80 mg (46%) of the desired product (logP (pH 2.3): 1.40); ¹H NMR (400 MHz, DMSO-d6) δ=9.40 (s, 1H), 7.91 (s, 1H), 7.81 (d, 1H), 7.72 (d, 1H), 7.10 (s, 1H), 3.56-3.58 (m, 4H), 2.85-2.87 (m, 1H), 1.90-1.92 (m, 4H), 0.76-0.80 (m, 2H), 0.64-0.67 (m, 2H).

### Preparation of Compounds of the Formula (I)

### 5-Chloro-N⁴-cyclopropyl-N²-2-methylthiophen-5-yl-2,4-diamine (Example 26, Scheme 8)

A mixture of 200 mg (0.98 mmol) of 5-chloro-2-chloro-N-cyclopropylpyrimidin-4-amine, 251 mg (1.17 mmol) of 2-(N-tert-butoxycarbonylamino)-5-methylthiophene and 149 mg (0.78 mmol) of 4-toluenesulphonic acid in 12 ml of dioxane is stirred at 105° C. for 18 h. After cooling, the reaction mixture is stirred into ice-water and extracted with ethyl acetate (3×100 ml). The combined organic phases are washed with 50 ml of saturated aq. NaHCO₃ and dried over Na₂SO₄, filtered and freed from the solvent under reduced pressure. The crude product is then purified by column chromatography (cyclohexane/ethyl acetate 1:1). This gives 275 mg (83%) of the desired product (logP (pH 2.3): 2.09); ¹H NMR (400 MHz, DMSO-d6) δ=7.87 (s, 1H), 6.94 (s, 1H), 6.43-6.47 (m, 2H), 2.96-3.00 (m, 1H), 2.31 (s, 3H), 0.74-0.80 (m, 2H), 0.67-0.69 (m, 2H).

### Preparation of Starting Materials of the Formula (Ix)

### 2-(Thiophen-3-ylamino)-5-chloropyrimidin-4(3H)-one (IX-1, Scheme 3)

A solution consisting of 3.27 ml of a 1M NaOH solution (aq.) and 1 ml of water is added to a solution of 500 mg (2.73 mmol) of 2,4,5-trichloropyrimidine in 10 ml of dioxane. After 4 d of stirring at room temperature, the reaction mixture is concentrated under reduced pressure. The residue is taken up in 50 ml of ethyl acetate and neutralized with 1 N HCl (aq.). The organic phase is separated off and then washed with 10 ml of water, dried over MgSO₄ and freed from the solvent under reduced pressure. The crude product, together with 445 mg (4.55 mmol) of 3-aminothiophene and 532 mg (3.09 mmol) of 4-toluenesulphonic acid, is taken up in 10 ml of dioxane and heated with stirring at 105° C. After 18 h, the reaction mixture is concentrated under reduced pressure and the residue is taken up in 50 ml of ethyl acetate. The organic phase is washed with 10 ml of saturated aq. NaHCO₃ and then with 10 ml of water, dried over MgSO₄ and freed from the solvent under reduced pressure. This gives 500 mg of 2-(thiophen-3-ylamino)-5-chloropyrimidin-4(3H)-one (IX-1) which is directly reacted further without further purification (logP (pH2.3): 1.38).

### Preparation of Starting Materials of the Formula (X, Scheme 4)

### 4,5-Dichloro-N-(3-thienyl)pyrimidin-2-amine (X-1)

A solution of 400 mg of 2-(thiophen-3-ylamino)-5-chloropyrimidin-4(3H)-one in 2 ml of phosphoryl chloride is heated at 95° C. for 18 h. After cooling, the reaction mixture is concentrated under reduced pressure, added to water and extracted with dichloromethane (3×20 ml). The combined organic phases are dried over MgSO₄ and freed from the solvent under reduced pressure. This gives 450 mg of 4,5-dichloro-N-(3-thienyl)pyrimidin-2-amine (X-1) (logP (pH 2.3): 3.55); ¹H NMR (400 MHz, DMSO-d6) δ=9.70 (s, 1H), 8.33 (s, 1H), 7.65-7.67 (m, 1H), 7.47-7.49 (m, 1 H), 7.41-7.43 (m, 1H).

### EXAMPLES

The compounds of the formula I listed in Table I below can be obtained analogously to the methods given above.

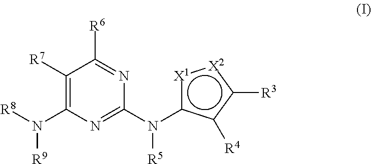

where X¹═S or CR¹

### X²═S or CR²

TABLE I

| Ex. | X¹ | R¹ | X² | R² | R³ | R⁴ | R⁵ | R⁶ | R⁷ | R⁸ | R⁹ | logp | [M + H]⁺ | 1H-NMR δ in ppm [d] |
|---|---|---|---|---|---|---|---|---|---|---|---|---|---|---|
| 1 | CR¹ | H | S | | H | H | H | H | chloro | H | cyclopropyl | 1.63[b] | 267 | 9.37 (s, 1 H), 7.89 (s, 1 H), |
| | | | | | | | | | | | | | | 7.64-7.63 (q, 1H), 7.34-7.32 |
| | | | | | | | | | | | | | | (q, 1H), 7.24-7.22 (q, 1H), 6.95 |
| | | | | | | | | | | | | | | (s, 1H), 2.89-2.84 (m, 1H), |
| | | | | | | | | | | | | | | 0.78-0.75 (m, 2H), 0.67-0.63 |
| | | | | | | | | | | | | | | (m, 2H). |
| 2 | CR¹ | H | S | | H | H | H | H | CF₃ | H | cyclopropyl | 2.69[b] | 301 | 9.03 (s, 1 H), 8.16 (s, 1 H), |
| | | | | | | | | | | | | | | 7.72 (d, 1 H), 7.36-7.38 (m, 1 H), |
| | | | | | | | | | | | | | | 7.28-7.29 (m, 1 H), 6.80 (s, 1 H), |
| | | | | | | | | | | | | | | 2.88-2.93 (m, 1 H), |
| | | | | | | | | | | | | | | 0.77-0.82 (m, 2 H), 0.66-0.69 |
| | | | | | | | | | | | | | | (m, 2 H). |
| 3 | CR¹ | H | S | | H | H | H | H | bromo | H | cyclobutyl | 2.12[b] | 326/328 | 9.30 (s, 1 H), 7.99 (s, 1 H), |
| | | | | | | | | | | | | | | 7.43-7.45 (m, 1 H), 7.33-7.35 |
| | | | | | | | | | | | | | | (m, 1 H), 7.18-7.20 (m, 1 H), 6.64 |
| | | | | | | | | | | | | | | (d, 1 H), 4.52-4.55 (m, 1 H), |
| | | | | | | | | | | | | | | 2.11-2.32 (m, 4 H), 1.68-1.75 |
| | | | | | | | | | | | | | | (m, 2 H). |
| 4 | CR¹ | H | S | | H | H | H | H | bromo | H | cyclopropyl | 1.71[b] | | 9.4 (s, 1 H), 7.98 (s, 1 H), |
| | | | | | | | | | | | | | | 7.64 (q, 1 H), 7.34-7.32 (q, 1 H), |
| | | | | | | | | | | | | | | 7.24-7.22 (q, 1 H), 6.72 (s, 1 H), |
| | | | | | | | | | | | | | | 2.89-2.80 (m, 1 H), |
| | | | | | | | | | | | | | | 0.79-0.73 (m, 2 H), 0.67-0.63 |
| | | | | | | | | | | | | | | (m, 2 H). |
| 5 | CR¹ | H | S | | methoxy- | H | H | H | chloro | H | cyclopropyl | 1.68[a] | 325 | 9.56 (s, 1 H), 8.07 (s, 1 H), |
| | | | | | carbonyl | | | | | | | | | 7.92 (s, 1 H), 7.86 (s, 1 H), 7.06 |
| | | | | | | | | | | | | | | (s, 1 H), 3.80 (s, 3 H), 2.83-2.86 |
| | | | | | | | | | | | | | | (m, 1 H), 0.79-0.84 (m, 2H), |
| | | | | | | | | | | | | | | 0.63-0.67 (m, 2H). |
| 6 | CR¹ | H | S | | pyrrolidin- | H | H | H | chloro | H | cyclopropyl | 1.42[a] | 364 | 9.40 (s, 1H), 7.91 (s, 1 H), |
| | | | | | 1-yl- | | | | | | | | | 7.81 (d, 1 H), 7.72 (d, 1 H), 7.10 |
| | | | | | carbonyl | | | | | | | | | (s, 1 H), 3.56-3.58 (m, 4 H), |
| | | | | | | | | | | | | | | 2.85-2.87 (m, 1 H), 1.90-1.92 |
| | | | | | | | | | | | | | | (m, 4 H), 0.76-0.80 (m, 2H), |
| | | | | | | | | | | | | | | 0.64-0.67 (m, 2H). |
| 7 | CR¹ | H | S | | piperidin- | H | H | H | chloro | H | cyclopropyl | 1.68[a] | 378 | 9.42 (s, 1H), 7.90 (s, 1 H), |
| | | | | | 1-yl- | | | | | | | | | 7.75 (d, 1 H), 7.48 (d, 1 H), 7.01 |
| | | | | | carbonyl | | | | | | | | | (s, 1 H), 3.57-3.65 (m, 4 H), |
| | | | | | | | | | | | | | | 2.83-2.87 (m, 1 H), 1.51-1.65 |
| | | | | | | | | | | | | | | (m, 6 H), 0.75-0.79 (m, 2H), |
| | | | | | | | | | | | | | | 0.64-0.67 (m, 2H). |
| 8 | CR¹ | H | S | | methyl- | H | H | H | chloro | H | cyclopropyl | 1.6[a] | 366 | 9.51 (s, 1 H), 7.93 (s, 1 H), |
| | | | | | (propan- | | | | | | | | | 7.76 (d, 1 H), 7.52 (d, 1 H), 7.16 |
| | | | | | 2-yl)- | | | | | | | | | (s, 1 H), 4.51-4.55 (m, 1 H), 2.91 |
| | | | | | carbamoyl | | | | | | | | | (s, 3 H), 2.58-2.59 (m, 1 H), 1.12 |
| | | | | | | | | | | | | | | (d, 6H), 0.77-0.79 (m, 2H), |
| | | | | | | | | | | | | | | 0.66-0.68 (m, 2H). |
| 9 | CR¹ | H | S | | diethyl- | H | H | H | chloro | H | cyclopropyl | 1.62[a] | 366 | 9.45 (s, 1H), 7.91 (s, 1 H), |
| | | | | | carbamoyl | | | | | | | | | 7.77 (d, 1 H), 7.52 (d, 1 H), 7.01 |
| | | | | | | | | | | | | | | (s, 1 H), 3.47 (q, 4 H), 2.84-2.87 |
| | | | | | | | | | | | | | | (m, 1 H), 1.17 (tr, 6 H), |
| | | | | | | | | | | | | | | 0.75-0.79 (m, 2H), 0.64-0.67 |
| | | | | | | | | | | | | | | (m, 2H). |
| 10 | S | | CR² | acetyl | H | H | H | H | chloro | H | cyclopropyl | 1.87[a] | 309 | 8.01 (s, 1 H), 7.64 (d, 1 H), |
| | | | | | | | | | | | | | | 7.24 (s, 1 H), 6.69 (d, 1 H), |
| | | | | | | | | | | | | | | 2.95-3.00 (m, 1 H), 2.37 (s, 3 H), |
| | | | | | | | | | | | | | | 0.84-0.88 (m, 2H), 0.70-0.73 |
| | | | | | | | | | | | | | | (m, 2H). |
| 11 | S | | CR² | (1E)-N- | H | H | H | H | chloro | H | cyclopropyl | 2.98[a] | 338 | main isomer: 7.95 (s, 1 H), |
| | | | | methoxy- | | | | | | | | | | 7.26 (d, 1 H), 7.21 (s, 1 H), 7.16 |
| | | | | ethanimi- | | | | | | | | | | (s, 1 H), 7.06 (d, 1 H), 3.81 |
| | | | | doyl | | | | | | | | | | (s, 3 H), 2.98-3.02 (m, 1 H), 2.16 |
| | | | | | | | | | | | | | | (s, 3 H), 0.80-0.89 (m, 2H), |
| | | | | | | | | | | | | | | 0.71-0.79 (m, 2H). |
| 12 | S | | CR² | H | cyano | H | H | H | chloro | H | cyclopropyl | 2.55[a] | 292 | 8.56 (d, 1 H), 8.03 (s, 1 H), |
| | | | | | | | | | | | | | | 7.60 (d, 1 H), 7.31 (s, 1 H), 6.69 |
| | | | | | | | | | | | | | | (d, 1 H), 2.94-3.02 (m, 1 H), |
| | | | | | | | | | | | | | | 0.80-0.86 (m, 2H), 0.71-0.74 |
| | | | | | | | | | | | | | | (m, 2H). |
| 13 | CR¹ | H | S | | acetyl | H | H | H | chloro | H | cyclopropyl | 1.44[a] | 309 | 8.00 (d, 1 H), 7.94 (d, 1 H), |
| | | | | | | | | | | | | | | 7.92 (s, 1 H), 7.05 (s, 1 H), 3.57 |
| | | | | | | | | | | | | | | (s, 3 H), 2.81-2.89 (m, 1 H), |
| | | | | | | | | | | | | | | 0.77-0.80 (m, 2H), 0.64-0.66 |
| | | | | | | | | | | | | | | (m, 2H). |
| 14 | CR¹ | H | S | | cyan | H | H | H | chloro | H | cyclopropyl | 2.23[a] | 292 | 9.68 (s, 1 H), 7.99 (m, 2 H), |
| | | | | | | | | | | | | | | 7.93 (s, 1 H), 7.09 (s, 1 H), |
| | | | | | | | | | | | | | | 2.83-2.87 (m, 1 H), 0.76-0.81 |
| | | | | | | | | | | | | | | (m, 2H), 0.63-0.67 (m, 2H). |
| 15 | S | | CR² | methoxy- | H | H | H | H | chloro | H | cyclopropyl | 2.25[a] | 325 | 8.01 (s, 1 H), 7.53 (d, 1 H), |
| | | | | carbonyl | | | | | | | | | | 7.24 (s, 1 H), 6.70 (d, 1 H), 3.73 |
| | | | | | | | | | | | | | | (s, 3 H), 2.95-3.00 (m, 1 H), |
| | | | | | | | | | | | | | | 0.83-0.87 (m, 2H), 0.70-0.74 |
| | | | | | | | | | | | | | | (m, 2H). |
| 16 | S | | CR² | cyan | H | H | H | H | chloro | H | cyclopropyl | 2.62[c]; | 292 | 8.55 (d, 1 H), 8.02 (s, 1 H), |
| | | | | | | | | | | | | 2.55[b] | | 7.60 (d, 1 H), 7.30 (s, 1 H), 6.69 |
| | | | | | | | | | | | | | | (d, 1 H), 2.94-3.04 (m, 1 H), |
| | | | | | | | | | | | | | | 0.82-0.89 (m, 2H), 0.71-0.74 |
| | | | | | | | | | | | | | | (m, 2H). |
| 17 | CR¹ | H | S | | H | H | H | H | chloro | H | 2-methyl- | 1.97[a] | 281 | main isomer: 9.32 (s, 1 H), |
| | | | | | | | | | | | cyclopropyl | | | 7.89 (s, 1 H), 7.62-7.63 (m, 1 H), |
| | | | | | | | | | | | | | | 7.32-7.34 (m, 1 H), |
| | | | | | | | | | | | | | | 7.22-7.24 (m, 1 H), 6.89 (s, 1 H), |
| | | | | | | | | | | | | | | 2.55-2.60 (m, 1 H), 1.12 (d, 3 H), |
| | | | | | | | | | | | | | | 0.94-0.95 (m, 1H), 0.84-0.89 |
| | | | | | | | | | | | | | | (m, 1H), 0.51-0.59 (m, 1 H). |
| 18 | CR¹ | H | S | | H | H | H | H | chloro | H | cyclobutyl | 3.46[c]; | 281 | 9.28 (s, 1 H), 7.90 (s, 1 H), |
| | | | | | | | | | | | | 1.96[b] | | 7.44-7.45 (m, 1 H), 7.33-7.35 |
| | | | | | | | | | | | | | | (m, 1 H), 7.18-7.20 (m, 1 H), 6.91 |
| | | | | | | | | | | | | | | (d, 1 H), 4.51-4.57 (m, 1 H), |
| | | | | | | | | | | | | | | 2.26-2.31 (m, 2 H), 2.09-2.19 |
| | | | | | | | | | | | | | | (m, 2H), 1.66-1.76 (m, 2H). |
| 19 | CR¹ | H | S | | mor- | H | H | H | chloro | H | cyclopropyl | 2.09[c]; | 380 | 9.40 (s, 1 H), 7.91 (s, 1 H), |
| | | | | | pholin- | | | | | | | 1.45[b] | | 7.78 (d, 1 H), 7.53 (d, 1 H), 7.02 |
| | | | | | 4-yl- | | | | | | | | | (s, 1 H), 3.63 (s, 8 H), 2.83-2.86 |
| | | | | | carbonyl | | | | | | | | | (m, 1 H), 0.75-0.79 (m, 2H), |
| | | | | | | | | | | | | | | 0.64-0.67 (m, 2H). |
| 20 | CR¹ | H | S | | tert- | H | H | H | chloro | H | cyclopropyl | 2.11[b] | 364 | 9.32 (s, 1 H), 8.05 (d, 1 H), |
| | | | | | butyl- | | | | | | | | | 7.92 (s, 1 H), 7.82 (d, 1 H), 7.05 |
| | | | | | carba- | | | | | | | | | (s, 1 H), 2.64-2.66 (m, 1 H), 1.35 |
| | | | | | moyl | | | | | | | | | (s, 9 H), 0.81-0.84 (m, 2H), |
| | | | | | | | | | | | | | | 0.63-0.67 (m, 2H). |
| 21 | CR¹ | H | S | | prop-2- | H | H | H | chloro | H | cyclopropyl | 2.28[c] | 350 | 9.45 (s, 1 H), 8.33 (s, 1 H), |
| | | | | | en-1- | | | | | | | | | 7.90 (s, 1 H), 7.78 (d, 1 H), 7.74 |
| | | | | | ylcarba- | | | | | | | | | (d, 1 H), 7.01 (s, 1 H), 5.85-5.90 |
| | | | | | moyl | | | | | | | | | (m, 1 H), 5.05-5.19 (m, 2 H), |
| | | | | | | | | | | | | | | 3.82-3.85 (m, 2 H), 2.85-2.92 |
| | | | | | | | | | | | | | | (m, 1 H), 0.75-0.78 (m, 2H), |
| | | | | | | | | | | | | | | 0.64-0.66 (m, 2H.) |
| 22 | CR¹ | H | S | | methyl- | H | H | H | chloro | H | cyclopropyl | 2.65[c]; | 364 | 9.42 (s, 1 H), 7.90 (s, 1 H), |
| | | | | | (prop- | | | | | | | 1.89[b] | | 7.79 (d, 1 H), 7.58 (d, 1 H), 7.00 |
| | | | | | 2-en-1- | | | | | | | | | (s, 1 H), 5.85-5.93 (m, 1 H), |
| | | | | | yl)carba- | | | | | | | | | 5.20-5.26 (m, 2 H), 4.08-4.10 |
| | | | | | moyl | | | | | | | | | (m, 2 H), 3.12 (s, 3 H), 2.86-2.90 |
| | | | | | | | | | | | | | | (m, 1 H), 0.73-0.78 (m, 2H), |
| | | | | | | | | | | | | | | 0.66-0.68 (m, 2H). |
| 23 | CR¹ | H | S | | (2- | H | H | H | chloro | H | cyclopropyl | 2.01[c]; | 366 | 9.50 (s, 1 H), 8.12 (s, 1 H), |
| | | | | | methoxy- | | | | | | | 1.38[b] | | 7.92 (s, 1 H), 7.77 (d, 1 H), 7.74 |
| | | | | | ethyl)- | | | | | | | | | (d, 1 H), 7.11 (s, 1 H), 3.60-3.64 |
| | | | | | carba- | | | | | | | | | (m, 2 H), 3.37-3.46 (m, 2 H), 3.29 |
| | | | | | moyl | | | | | | | | | (s, 3 H), 2.88-2.92 (m, 1 H), |
| | | | | | | | | | | | | | | 0.77-0.79 (m, 2H), 0.65-0.67 |
| | | | | | | | | | | | | | | (m, 2H). |
| 24 | CR¹ | H | S | | (1- | H | H | H | chloro | H | cyclopropyl | 2.22 [c]; | 382 | 9.69 (s, 1 H), 7.97 (s, 1 H), |
| | | | | | Methoxy- | | | | | | | 1.58[b] | | 7.83 (d, 1 H), 7.73 (d, 1 H), 7.48 |
| | | | | | propan- | | | | | | | | | (s, 1 H), 4.62-4.12 (m, 1 H), |
| | | | | | 2-yl)- | | | | | | | | | 3.60-3.62 (m, 1 H), 3.38-3.41 |
| | | | | | carbamoyl | | | | | | | | | (m, 1 H), 3.28 (s, 3 H), 3.01-3.05 |
| | | | | | | | | | | | | | | (m, 1 H), 1.12 (d, 3 H), 0.78-0.82 |
| | | | | | | | | | | | | | | (m, 2H), 0.58-0.62 (m, 2H). |
| 25 | CR¹ | H | S | | hydroxy- | H | H | H | chloro | H | cyclopropyl | 2.96[c]; | 325 | 8.06 (d, 1 H), 7.92 (s, 1 H), |
| | | | | | carbamoyl | | | | | | | 2.1[b] | | 7.86 (d, 1 H), 7.05 (s, 1 H), 3.80 |
| | | | | | | | | | | | | | | (s, 1 H), 2.83-2.89 (m, 1 H), |
| | | | | | | | | | | | | | | 0.80-0.83 (m, 2H), 0.64-0.66 |
| | | | | | | | | | | | | | | (m, 2H). |
| 26 | S | | CR² | CH₃ | H | H | H | H | chloro | H | cyclopropyl | 3.20[c]; | 281 | 7.87 (s, 1 H), 6.94 (s, 1 H), |
| | | | | | | | | | | | | 2.09[b] | | 6.43-6.47 (m, 2 H), 2.96-3.00 |
| | | | | | | | | | | | | | | (m, 1 H), 2.31 (s, 3 H), 0.74-0.80 |
| | | | | | | | | | | | | | | (m, 2H), 0.67-0.69 (m, 2H). |
| 27 | CR¹ | H | S | | H | H | H | H | chloro | H | propan-2-yl | 3.28[c]; | 269 | 9.29 (s, 1 H), 7.89 (s, 1 H), |
| | | | | | | | | | | | | 1.66[b] | | 7.42-7.43 (m, 1 H), 7.32-7.34 |
| | | | | | | | | | | | | | | (m, 1 H), 7.18-7.20 (m, 1 H), 6.40 |
| | | | | | | | | | | | | | | (d, 1 H), 4.30-4.34 (m, 1 H), |
| | | | | | | | | | | | | | | 1.24 (d, 6 H). |
| 28 | CR¹ | H | S | | H | H | H | H | bromo | H | propan-2-yl | 3.43[c]; | 314/316 | 9.31 (s, 1 H), 7.98 (s, 1 H), |
| | | | | | | | | | | | | 1.85[b] | | 7.41-7.42 (m, 1 H), 7.33-7.35 |
| | | | | | | | | | | | | | | (m, 1 H), 7.18-7.20 (m, 1 H), 6.12 |
| | | | | | | | | | | | | | | (d, 1 H), 4.27-4.33 (m, 1 H), |
| | | | | | | | | | | | | | | 1.24 (d, 6 H). |
| 29 | CR¹ | H | S | | H | H | H | H | bromo | H | 2,2,2- | 3.50[c] | 354/356 | 9.42 (s, 1 H), 8.70 (s, 1 H), |
| | | | | | | | | | | | trifluoro- | | | 8.18 (s, 1 H), 7.38-7.39 (m, 1 H), |
| | | | | | | | | | | | ethyl | | | 7.34-7.35 (m, 1 H), |
| | | | | | | | | | | | | | | 7.15-7.16 (m, 1 H), 4.11-4.13 |
| | | | | | | | | | | | | | | (m, 2 H). |
| 30 | CR¹ | H | S | | H | H | H | H | bromo | H | 2,2- | 2.83[c]; | 336/338 | 9.41 (s, 1 H), 8.06 (s, 1 H), |
| | | | | | | | | | | | difluoro- | 1.99[b] | | 7.38-7.39 (m, 1 H), 7.34-7.36 |
| | | | | | | | | | | | ethyl | | | (m, 1 H), 7.17-7.19 (m, 1 H), 7.05 |
| | | | | | | | | | | | | | | (s, 1 H), 6.05-6.35 (m, 1 H), |
| | | | | | | | | | | | | | | 3.79-3.83 (m, 2 H). |
| 31 | CR¹ | H | S | | H | H | H | H | chloro | H | 2,2- | 2.72[c]; | 291 | 9.39 (s, 1 H), 7.98 (s, 1 H), |
| | | | | | | | | | | | difluoroethyl | 1.88[b] | | 7.39-7.40 (m, 1 H), 7.23 (s, 1 H), |
| | | | | | | | | | | | | | | 7.34-7.36 (m, 1 H), |
| | | | | | | | | | | | | | | 7.17-7.19 (m, 1 H), 6.05-6.33 |
| | | | | | | | | | | | | | | (m, 1 H), 3.80-3.83 (m, 2 H). |
| 32 | CR¹ | H | S | | H | H | H | H | chloro | H | cyclopropyl | 3.31[c]; | 281 | 9.28 (s, 1 H), 7.90 (s, 1 H), |
| | | | | | | | | | | | methyl | 2.33[b] | | 7.43-7.45 (m, 1 H), 7.32-7.34 |
| | | | | | | | | | | | | | | (m, 1 H), 7.18-7.20 (m, 1 H), 6.92 |
| | | | | | | | | | | | | | | (s, 1 H), 3.24-3.33 (m, 2 H), |
| | | | | | | | | | | | | | | 1.13-1.18 (m, 1 H), 0.41-0.45 |
| | | | | | | | | | | | | | | (m, 2 H), 0.26-0.29 (m, 2 H). |
| 33 | S | | CR² | methyl- | H | H | H | H | chloro | H | cyclopropyl | 2.02[c]; | 343 | 7.98 (s, 1 H), 7.43 (d, 1 H), |
| | | | | sulphonyl | | | | | | | | 1.97[b] | | 7.09 (s, 1 H), 6.61 (d, 1 H), 3.57 |
| | | | | | | | | | | | | | | (s, 3 H), 2.93-2.98 (m, 1 H), |
| | | | | | | | | | | | | | | 0.79-0.86 (m, 2H), 0.72-0.81 |
| | | | | | | | | | | | | | | (m, 2H). |
| 34 | S | | CR² | methyl- | H | H | H | H | chloro | H | cyclopropyl | 3.36[c]; | 313 | 7.93 (s, 1 H), 7.08 (s, 1 H), |
| | | | | sulphanyl | | | | | | | | 2.84[b] | | 6.88 (d, 1 H), 6.53 (d, 1 H), |
| | | | | | | | | | | | | | | 2.93-2.98 (m, 1 H), 2.35 (s, 3 H), |
| | | | | | | | | | | | | | | 0.78-0.83 (m, 2H), 0.69-0.71 |
| | | | | | | | | | | | | | | (m, 2H). |
| 35 | S | | CR² | methyl- | H | H | H | H | bromo | H | cyclopropyl | 3.53[c]; | 358/360 | 8.01 (s, 1 H), 6.88 (d, 1 H), |
| | | | | sulphanyl | | | | | | | | 3.02[b] | | 6.82 (s, 1 H), 6.53 (d, 1 H), |
| | | | | | | | | | | | | | | 2.93-2.98 (m, 1 H), 2.35 (s, 3 H), |
| | | | | | | | | | | | | | | 0.78-0.82 (m, 2H), 0.69-0.71 |
| | | | | | | | | | | | | | | (m, 2H). |
| 36 | S | | CR² | CH₃ | H | H | H | H | CF₃ | H | cyclopropyl | 3.56[c]; | 315 | 8.14 (s, 1 H), 6.76 (s, 1 H), |
| | | | | | | | | | | | | 3.22[b] | | 6.56 (d, 1 H), 6.48 (d, 1 H), 3.56 |
| | | | | | | | | | | | | | | (s, 3 H), 3.03-3.05 (m, 1 H), |
| | | | | | | | | | | | | | | 0.78-0.82 (m, 2H), 0.68-0.71 |
| | | | | | | | | | | | | | | (m, 2H). |
| 37 | CR¹ | H | S | | hydrox- | H | H | H | Cl | H | cyclopropyl | 0.74[c]; | 311 | 8.02 (s, 1 H), 7.99 (d, 1 H), |
| | | | | | carbonyl | | | | | | | 1.30[b] | | 7.83 (d, 1 H), 7.69 (s, 1 H), 3.44 |
| | | | | | | | | | | | | | | (s, 1 H), 2.84-2.94 (m, 1 H), |
| | | | | | | | | | | | | | | 0.83-0.88 (m, 2H), 0.71-0.73 |
| | | | | | | | | | | | | | | (m, 2H). |
| 38 | CR¹ | H | S | | methyl- | H | H | H | Cl | H | cyclopropyl | 2.86[c]; | 378 | 7.96 (s, 1 H), 7.78 (d, 1 H), |
| | | | | | (cyclo- | | | | | | | 2.08[b] | | 7.58 (d, 1 H), 3.61-3.66 (m, 1 H), |
| | | | | | propyl- | | | | | | | | | 3.13 (s, 3 H), 3.11-3.15 (m, 2 H), |
| | | | | | methyl)- | | | | | | | | | 0.81-0.83 (m, 2 H), |
| | | | | | carbamoyl | | | | | | | | | 0.71-0.75 (m, 2H), 0.50-0.55 |
| | | | | | | | | | | | | | | (m, 2H), 0.23-0.26 (m, 2 H). |
| 39 | CR¹ | H | S | | pyrrolidin- | H | H | H | Cl | H | cyclopropyl | 3.23[c]; | 380 | 9.36 (s, 1 H), 7.91 (s, 1 H), |
| | | | | | 1- | | | | | | | 2.31[b] | | 7.78 (d, 1 H), 7.56 (d, 1 H), 7.04 |
| | | | | | ylthio- | | | | | | | | | (s, 1 H), 3.83-3.86 (m, 2 H), |
| | | | | | carbonyl | | | | | | | | | 3.61-3.66 (m, 2 H), 2.85-2.88 |
| | | | | | | | | | | | | | | (m, 1 H), 1.96-2.00 (m, 2 H), |
| | | | | | | | | | | | | | | 0.75-0.78 (m, 2H), 0.65-0.67 |
| | | | | | | | | | | | | | | (m, 2H). |
| 40 | CR¹ | H | S | | diethyl- | H | H | H | Cl | H | cyclopropyl | 3.51[c]; | 382 | 9.42 (s, 1 H), 7.91 (s, 1 H), |
| | | | | | thio- | | | | | | | 2.55[b] | | 7.70 (d, 1 H), 7.31 (d, 1 H), 7.03 |
| | | | | | carba- | | | | | | | | | (s, 1 H), 3.72-3.85 (m, 4 H), |
| | | | | | moyl | | | | | | | | | 2.83-2.86 (m, 1 H), 1.26 (tr, 6 H), |
| | | | | | | | | | | | | | | 0.75-0.78 (m, 2H), 0.64-0.67 |
| | | | | | | | | | | | | | | (m, 2H). |
| 41 | CR¹ | H | S | | chloro | H | H | H | Cl | H | cyclopropyl | 2.63[b] | 302 | 9.41 (s, 1 H), 7.90 (s, 1 H), |
| | | | | | | | | | | | | | | 7.42 (d, 1 H), 7.26 (d, 1 H), 6.98 |
| | | | | | | | | | | | | | | (s, 1 H), 2.79-2.83 (m, 1 H), |
| | | | | | | | | | | | | | | 0.76-0.79 (m, 2H), 0.51-0.53 |
| | | | | | | | | | | | | | | (m, 2H). |
| 42 | CR¹ | H | S | | H | H | H | H | Cl | H | 2-(trifluoro- | | 335 | |
| | | | | | | | | | | | methyl)- | | | |
| | | | | | | | | | | | cyclopropyl | | | |
| 43 | S | | CR² | chloro | H | H | H | H | Cl | H | cyclopropyl | 3.54[c]; | 302 | 7.93 (s, 1 H), 7.10 (s, 1 H), |
| | | | | | | | | | | | | 3.08[b] | | 6.76 (d, 1 H), 6.44 (d, 1 H), |
| | | | | | | | | | | | | | | 2.93-2.98 (m, 1 H), 2.31 (s, 3 H), |
| | | | | | | | | | | | | | | 0.71-0.82 (m, 2H), 0.67-0.69 |
| | | | | | | | | | | | | | | (m, 2H). |
| 44 | S | | CR² | methyl | H | H | H | H | Br | H | cyclopropyl | 3.35[c]; | 326/328 | 7.96 (s, 1 H), 6.69 (s, 1 H), |
| | | | | | | | | | | | | 2.27[b] | | 6.43-6.47 (m, 2 H), 2.95-3.00 |
| | | | | | | | | | | | | | | (m, 1 H), 0.72-0.80 (m, 2H), |
| | | | | | | | | | | | | | | 0.65-0.69 (m, 2H). |
| 45 | S | | CR² | methyl | H | H | H | H | H | H | cyclopropyl | 1.32[b] | 247 | 7.96 (d, 1 H), 7.83 (d, 1 H), |
| | | | | | | | | | | | | | | 6.42 (s, 1 H), 5.96-5.99 (m, 2 H), |
| | | | | | | | | | | | | | | 2.65-2.67 (m, 1 H), 2.30 (s, 3 H), |
| | | | | | | | | | | | | | | 0.71-0.76 (m, 2H), 0.49-0.52 |
| | | | | | | | | | | | | | | (m, 2H). |

The logP values were determined in accordance with EEC directive 79/831 Annex V.A8 by HPLC (High Performance Liquid Chromatography) on reversed-phase columns (C 18), using the methods below:
[a]The determination was carried out in the acidic range at pH 2.3 using the mobile phases 0.1% aqueous phosphoric acid and acetonitrile, linear gradient from 10% acetonitrile to 95% acetonitrile.
[b]The LC-MS determination in the acidic range was carried out at pH 2.7 using the mobile phases 0.1% aqueous formic acid and acetonitrile (contains 0.1% formic acid), linear gradient from 10% acetonitrile to 95% acetonitrile.
[c]The LC-MS determination in the neutral range was carried out at pH 7.8 using the mobile phases 0.001 molar aqueous ammonium bicarbonate solution and acetonitrile, linear gradient from 10% acetonitrile to 95% acetonitrile.
Calibration was carried out using unbranched alkan-2-ones (having 3 to 16 carbon atoms), with known logP values (determination of the logP values by the etention times using linear interpolation between two successive alkanones). The lambda-maX values were determined in the maxima of the chromatographic signals using the UV spectra from 200 nm to 400 nm.
[d]The chemical NMR shifts in ppm were measured at 400 MHz, unless indicated otherwise in the solvent DMSO-d₆ using tetramethylsilane as internal standard.
The following abbreviations describe signal splitting:
s = singlet,
d = doublet,
t = triplet,
q = quadruplet,
m = multiplet

### Use Examples

### Example A

### Venturia Test (Apples)/Protective

Solvents: 24.5 parts by weight of acetone
   24.5 parts by weight of dimethylacetamide
Emulsifier: 1 part by weight of alkylaryl polyglycol ether

To produce a suitable preparation of active compound, 1 part by weight of active compound is mixed with the stated amounts of solvents and emulsifier, and the concentrate is diluted with water to the desired concentration.

To test for protective activity, young plants are sprayed with the preparation of active compound at the stated application rate. After the spray coating is dried on, the plants are inoculated with an aqueous conidia suspension of the apple pathogen Venturia inaequalis and then remain in an incubation cabainet at about 20° C. and 100% relative atmospheric humidity for one day.

The plants are then placed in a greenhouse at 21° C. and a relative atmospheric humidity of about 90%.

Evaluation is carried out at 10 days after the inoculation. 0% means an efficacy which corresponds to that of the control, whereas an efficacy of 100% means that no infection is observed.

In this test, the compounds Nos. 1, 2, 4, 7, 8, 9, 17 and 18 from Table I show, at an active compound concentration of 100 ppm, an efficacy of 70% or more.

### Example B

### Botrytis Test (Bean)/Protective

Solvents: 24.5 parts by weight of acetone
   24.5 parts by weight of dimethylacetamide
Emulsifier: 1 part by weight of alkylaryl polyglycol ether

To produce a suitable preparation of active compound, 1 part by weight of active compound is mixed with the stated amounts of solvents and emulsifier, and the concentrate is diluted with water to the desired concentration.

To test for protective activity, young plants are sprayed with the preparation of active compound at the stated application rate. After the spray coating has dried on, two small pieces of agar colonized by Botrytis cinerea are placed onto each leaf. The inoculated plants are placed in a dark chamber at about 20° C. and 100% relative atmospheric humidity.

The size of the infected areas on the leaves is evaluated two days after the inoculation. 0% means an efficacy which corresponds to that of the control, whereas an efficacy of 100% means that no infection is observed.

In this test, the compounds Nos. 2, 4, 7, 8, 17 and 18 from Table I show, at an active compound concentration of 100 ppm, an efficacy of 70% or more.

### Example C

### Sphaerotheca Test (Cucumber)/Protective

Solvent: 49 parts by weight of an N,N-dimethylformamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young cucumber plants are sprayed with the preparation of active compound at the stated application rate. One day after the treatment, the plants are inoculated with a spore suspension of Sphaerotheca fuliginea. The plants are then placed in a greenhouse at 70% relative atmospheric humidity and a temperature of 23° C.

Evaluation is carried out seven days after the inoculation. 0% means an efficacy which corresponds to that of the control, whereas an efficacy of 100% means that no infection is observed.

In this test, the examples Nos. 1, 2, 4, 7, 8, 9, 36 and 40 from Table I show, at an active compound concentration of 500 ppm, an efficacy of 70% or more.

### Example D

### Alternaria Test (Tomato)/Protective

Solvent: 49 parts by weight of N,N-dimethylformamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young tomato plants are sprayed with the preparation of active compound at the stated application rate. One day after the treatment, the plants are inoculated with a spore suspension of Alternaria solani and then remain at 100% rel. humidity and 22° C. for 24 h. The plants then remain at 96% rd. atmospheric humidity and a temperature of 20° C.

Evaluation is carried out seven days after the inoculation. 0% means an efficacy which corresponds to that of the control, whereas an efficacy of 100% means that no infection is observed.

In this test, the examples Nos. 1, 6, 7, 8, 9, 13, 14, 17, 18, 19, 21, 22, 24, 26 and 38 from Table I show, at an active compound concentration of 500 ppm, an efficacy of 70% or more.

### Example E

### Leptosphaeria Nodorum Test (Wheat)/Protective

Solvent: 49 parts by weight of N,N-dimethylformamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concenrtration.

To test for protective activity, young wheat plants are sprayed with the preparation of active compound at the stated application rate. One day after the treatment, the plants are inoculated with an aqueous spore suspension of Leptosphaeria nodorum and then remain at 100% rd. atmospheric humidity and 22° C. for 48 h. The plants are then placed in a greenhouse at 90% rel. atmospheric humidity and a temperature of 22° C.

Evaluation is carried out 7-9 days after the inoculation. 0% means an efficacy which corresponds to that of the control, whereas an efficacy of 100% means that no infection is observed.

In this test, the examples Nos. 1, 2, 3, 4, 6, 7, 8, 9, 13, 14, 17, 18, 19, 21, 22, 23, 24, 26, 27, 28, 30, 31, 32, 36, 38, 39, 40 and 41 from Table I show, at an active compound concentration of 500 ppm, an efficacy of 70% or more.

### Example F

### Septoria Tritici Test (Wheat)/Protective

Solvent: 50 parts by weight of N,N-dimethylacetamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are sprayed with a spore suspension of Septoria tritici. The plants remain in an incubation cabin at 20° C. and 100% relative atmospheric humidity for 48 hours. The plants are then placed under a translucent hood at 15° C. and 100% relative atmospheric humidity for a further 60 hours.

The plants are placed in a greenhouse at a temperature of about 15° C. and a relative atmospheric humidity of 80%.

Evaluation is carried out 21 days after the inoculation. 0% means an efficacy which corresponds to that of the control, whereas an efficacy of 100% means that no infection is observed.

In this test, the examples Nos. 1, 2, 4, 7, 8, 9, 13, 18 and 40 from Table I show, at an active compound concentration of 500 ppm, an efficacy of 70% or more.

### Example G

### Pyricularia Test (Rice)/Protective

Solvent: 28.5 parts by weight of acetone
Emulsifier: 1.5 parts by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the preparation of active compound at the stated application rate. One day after the treatment, the plants are inoculated with an aqueous spore suspension of Pyricularia oryzae. The plants are then placed in a greenhouse at 100% relative atmospheric humidity and 25° C.

Evaluation is carried out five days after the inoculation. 0% means an efficacy which corresponds to that of the control, whereas an efficacy of 100% means that no infection is observed.

In this test, the compounds according to the invention Nos. 2, 3, 4, 9, 18, 19 and 27 from Table I show, at an active compound concentration of 250 ppm, an efficacy of 80% or more.

### Example H

### Rhizoctonia Test (Rice)/Protective

Solvent: 28.5 parts by weight of acetone
Emulsifier: 1.5 parts by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the preparation of active compound at the stated application rate. One day after the treatment, the plants are inoculated with hyphae of Rhizoctonia solani. The plants are then placed in a greenhouse at 100% relative atmospheric humidity and 25° C.

Evaluation is carried out four days after the inoculation. 0% means an efficacy which corresponds to that of the control, whereas an efficacy of 100% means that no infection is observed.

In this test, the examples Nos. 2, 3 and 4 from Table I show, at an active compound concentration of 250 ppm, an efficacy of 80% or more.

### Beispiel I

### Cochliobolus Test (Rice)/Protective

Solvent: 28.5 parts by weight of acetone
Emulsifier: 1.5 parts by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the preparation of active compound at the stated application rate. One day after the treatment, the plants are inoculated with an aqueous spore suspension of Cochliobolus miyabeanus. The plants are then placed in a greenhouse at 100% relative atmospheric humidity and 25° C.

Evaluation is carried out four days after the inoculation. 0% means an efficacy which corresponds to that of the control, whereas an efficacy of 100% means that no infection is observed.

In this test, the examples Nos. 2, 3, 4 and 18 from Table I show, at an active compound concentration of 250 ppm, an efficacy of 80% or more.

### Beispiel J

### Gibberella Test (Rice)/Protective

Solvent: 28.5 parts by weight of acetone
Emulsifier: 1.5 parts by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the preparation of active compound at the stated application rate. One day after the treatment, the plants are inoculated with an aqueous spore suspension of Gibberella zeae. The plants are then placed in a greenhouse at 100% relative atmospheric humidity and 25° C.

Evaluation is carried out five days after the inoculation. 0% means an efficacy which corresponds to that of the control, whereas an efficacy of 100% means that no infection is observed.

In this test, the example No. 2 from Table I shows, at an active compound concentration of 250 ppm, an efficacy of 80% or more.

### Example K

### Production of Fumonisin FB1 by Fusarium Proliferatum

The method used was adapted to microtitre plates using the method described by Lopez-Errasquin et al.: Journal of Microbiological Methods 68 (2007) 312-317.

Fumonisin-inducing liquid medium (Jimenez et al., Int. J. Food Microbiol. (2003), 89, 185-193) was inoculated with a concentrated spore suspension of Fusarium proliferatum (350 000 spores/ml, stored at −160° C.) to a final concentration of 2000 spores/ml.

The compounds were dissolved (10 mM in 100% DMSO) and diluted to 100 μM in H₂O. The compounds were tested at 7 concentrations in a range of from 50 μM to 0.01 μM (diluted, starting with the 100 μM stock solution in 10% DMSO).

From each diluted solution, 5 μl were mixed with 95 μA of inoculated medium in a well of a 96-well microarray plate. The plate was covered and incubated at 20° C. for 6 days.

At the beginning and after 6 days, an OD measurement (OD620 multiple read per well (square: 3×3)) was carried out to calculate the “pI50” growth.

After 6 days, a sample of the liquid medium was taken and diluted in 10% acetonitrile. The concentration of FB1 in the diluted samples was analysed by HPLC-MS/MS, and the results were used to calculate the “pI50 FB1” values.

HPLC-MS/MS was carried out using the parameters below:
Mass spectrometry instrument: Applied Biosystems API4000 QTrap

### HPLC: Agilent 1100

### Autosampler: CTC HTS PAL

Chromatography column: Waters Atlantis T3 (50×2 mm)
Examples of the Measured p150 Values

### Production of Fumonisin FB1 by Fusarium Proliferatum

| Example No. | pI50 | pI50 |
|---|---|---|
| from Table I | Fum | growth |
| 1 | 6.4 | 5.8 |
| 2 | 6.6 | 5.8 |
| 3 | 6.2 | 5.8 |
| 4 | 6.6 | 6.3 |
| 5 | 5.3 | 4.4 |
| 6 | 5.9 | 5.6 |
| 7 | 6.4 | 5.7 |
| 8 | 5.7 | 5.1 |
| 9 | 6.0 | 5.5 |
| 12 | 6.6 | 5.6 |

### Example L

### Production of DON/Acetyl-DON by Fusarium Graminearum

The compounds were tested in microtitre plates at 7 concentrations of from 0.07 μM to 50 μM in a DON-inducing liquid medium (1 g of (NH₄)₂HPO₄, 0.2 g of MgSO₄×7 H₂O, 3 g of KH₂PO₄, 10 g of glycerol, 5 g of NaCl and 40 g of sucrose per litre) with oat extract (10%) and DMSO (0.5%). Inoculation was carried out using a concentrated spore suspension of Fusarium graminearum at a final concentration of 2000 spores/ml.

The plate was incubated at high atmospheric humidity at 28° C. for 7 days.

At the beginning and after 3 days, an OD measurement was carried out at OD520 (repeated measurements: 3×3 measurements per well) to calculate the growth inhibition.

After 7 days, 100 μl of an 84/16 acetonitrile/water mixture were added, and a sample of the liquid medium were then taken from each well and diluted 1:100 in 10% acetonitrile. The proportions of DON and acetyl-DON in the samples were analysed by HPLC-MS/MS, and the measured values were used to calculate the inhibition of the DON/AcDON production compared to an active compound-free control.

The HPLC-MS/MS measurements were carried out using the parameters below:
Ionization type: ESI negative
Ion spray voltage: −4500 V
Spray gas temperature: 500° C.
Decluster potential: −40 V
Collision energy: −22 eV

### Collision gas: N₂

NMR trace: 355.0>264.9;
HPLC column: Waters Atlantis T3 (trifunctional C18 bondung, capped)
Particle size: 3 μm
Column dimensions: 50×2 mm

### Temperature: 40° C.

Solvent A: water/2.5 mM NH₄OAc+0.05% CH₃COOH (v/v)
Solvent B: methanol/2.5 mM NH₄OAc+0.05% CH₃COOH (v/v)
Flow rate: 400 μl/minute
Injection volume: 11 μl

### Gradient:

| Time [min] | A % | B % |
|---|---|---|
| 0 | 100 | 0 |
| 0.75 | 100 | 0 |
| 1.5 | 5 | 95 |
| 4 | 5 | 95 |
| 5 | 100 | 0 |
| 10 | 100 | 0 |

### Examples of DON Inhibition

The examples Nos. 5 and 9 showed an activity >80% in the inhibition of DON/AcDON at 50 μM. The inhibition of growth of Fusarium graminearum

by the examples having an activity >80% varied from 80 to 100% at 50 μM.

1. A compound of formula (I) 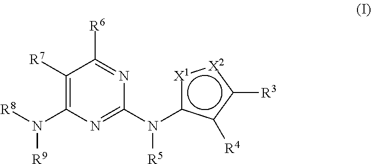
wherein
X¹ represents sulphur or CR¹,
X² represents sulphur or CR²,
where exactly one of the radicals X¹ and X² represents a sulphur atom,
R¹ represents hydrogen, C₁-C₄-alkyl, C₁-C₄-alkoxy or halogen,
if X² represents CR²,
R² and R³ independently of one another represent hydrogen, halogen, CN, nitro, OR¹⁰, O(CH₂)ₘOR¹⁰, O[C(R¹⁰)₂]ₘOR¹⁰, O[C(R¹⁰)₂]ₘN(R¹⁰)₂, OCOR¹¹, SR¹⁰, SOR¹⁰, SO₂R¹⁰, C═OR¹⁰, CH═NOR¹⁰, CR¹¹═NOR¹⁰, COCl, CON(R¹⁰)₂, COOR¹⁰, NR¹⁰COR¹⁰, N(R¹⁰)₂, [C(R¹⁰)₂]ₘCN, (CH₂)ₘOR¹⁰, (CH₂)ₘSR¹⁰, [C(R¹⁰)₂]ₘSR¹⁰, (CH₂)ₘSOR¹⁰, (CH₂)ₘSO₂R¹⁰, (CH₂)ₘN(R¹⁰)₂, [C(R¹⁰)₂]ₘN(R¹⁰)₂, (CH₂)ₘCOR¹⁰, [C(R¹⁰)₂]ₘCOR¹⁰, unsubstituted or substituted C₁-C₈-alkyl, C₂-C₆-alkenyl or C₁-C₈-haloalkyl;
where m=1-4,
where the substituents independently of one another are selected from the group consisting of: hydrogen, fluorine, chlorine or bromine, C₁-C₄-alkyl, C₁-C₄-alkoxy, hydroxyl, oxo, C₁-C₄-haloalkyl and cyano,
where, if X² represents a sulphur atom, the above definitions apply only to R³,
R⁴ represents hydrogen, C₁-C₄-alkyl, C₁-C₄-alkoxy or halogen,
R⁵ represents hydrogen, C₁-C₂-alkyl, C₁-C₄-alkoxy-C₁-C₄-alkyl, C₁-C₄-trialkylsilyl, C₁-C₄-trialkylsilylethyl, C₁-C₄-dialkylmonophenylsilyl, CHO, (C₁₋₄-alkyl)carbonyl, (C₁-C₄-alkoxy-C₁-C₄-alkyl)carbonyl, (C₃-C₆-alkenyloxy)carbonyl, (C₃-C₆-cycloalkyl)carbonyl, (halo-C₁-C₄-alkoxy-C₁-C₄-alkyl)carbonyl, (C₁-C₄-haloalkyl)carbonyl, (C₁-C₄-alkoxy)carbonyl, (C₁-C₄-haloalkoxy)carbonyl, benzyloxycarbonyl, unsubstituted or substituted benzyl, unsubstituted or substituted C₂-C₆-alkenyl, unsubstituted or substituted C₂-C₆-alkynyl, C₁-C₂-alkylsulfinyl or C₁-C₂-alkylsulphonyl,
where the substituents independently of one another are selected from the group consisting of: hydrogen, halogen, nitro, C₁-C₄-alkyl, C₁-C₄-alkoxy, hydroxyl, C₁-C₄-haloalkyl of and cyano,
R⁶ represents hydrogen, C₁-C₃-alkyl, cyano or C₁-C₃-haloalkyl,
R⁷ represents halogen, cyano, C₁-C₃-alkyl, C₁-C₃-haloalkyl, C₁-C₃-haloalkyloxy, SMe, SOMe or SO₂Me,
R⁸ represents hydrogen, C₁-C₂-alkyl, C₁-C₄-alkoxy-C₁-C₄-alkyl, C₁-C₆-trialkylsilyl, C₁-C₄-trialkylsilylethyl, C₁-C₄-dialkylmonophenylsilyl, (C₁-C₄-alkyl)carbonyl, (C₁-C₄-haloalkyl)carbonyl, (C₁-C₄-alkoxy)carbonyl, unsubstituted or substituted benzyl, unsubstituted or substituted C₂-C₆-alkenyl, unsubstituted or substituted C₂-C₆-alkynyl, C₁-C₆-alkylsulphinyl, C₁-C₆-alkylsulphonyl, C₁-C₆-haloalkylsulphinyl or C₁-C₆-haloalkylsulphonyl,
where the substituents independently of one another are selected from the group consisting of: fluorine, chlorine and bromine atoms, cyano, hydroxyl, methoxy and CF₃,
R⁹ represents straight-chain or branched unsubstituted or substituted C₁-C₇-alkyl, straight-chain or branched unsubstituted or substituted C₂-C₇-haloalkyl, unsubstituted or substituted C₃-C₇-cycloalkyl, straight-chain or branched unsubstituted or substituted C₃-C₇-cycloalkyl-C₁-C₃-alkyl, straight-chain or branched unsubstituted or substituted C₃-C₇-alkenyl, straight-chain or branched unsubstituted or substituted C₃-C₇-alkynyl, straight-chain or branched unsubstituted or substituted C₁-C₄-alkoxy-C₁-C₄-alkyl, straight-chain or branched unsubstituted or substituted C₁-C₄-haloalkoxy-C₁-C₄-alkyl, 2-methyl-1-(methylsulphanyl)propan-2-yl or oxetan-3-yl,
or
R⁸ and R⁹ together with the nitrogen atom to which they are attached form an unsubstituted or substituted 3- to 7-membered saturated cycle which may contain up to one further heteroatom selected from the group consisting of oxygen, sulphur and nitrogen,
R¹⁰ are identical or different and represent hydrogen, C₁-C₆-alkyl, C₁-C₆-haloalkyl, unsubstituted or substituted C₃-C₆-cycloalkyl, C₁-C₄-trialkylsilyl, unsubstituted or substituted C₂-C₄-alkenyl, unsubstituted or substituted C₂-C₄-alkynyl, unsubstituted or substituted phenyl, C₁-C₄-alkoxy-C₁-C₄-alkyl, C₁-C₄-alkylthio-C₁-C₄-alkyl, unsubstituted or substituted benzyl or a 3- to 7-membered unsubstituted or substituted saturated or unsaturated cycle which may contain no or up to four heteroatoms selected from the group consisting of N, O and S, where two oxygen atoms are not adjacent,
or,
if two radicals R¹⁰ are attached to a nitrogen atom, two radicals R¹⁰ may form a 3- to 7-membered unsubstituted or substituted saturated or unsaturated cycle which may contain up to four further heteroatoms selected from the group consisting of N, O and S, where two oxygen atoms are not adjacent,
or
if two radicals R¹⁰ are adjacent in the grouping NR¹⁰COR¹⁰, two radicals R¹⁰ may form a 3- to 7-membered unsubstituted or substituted saturated or unsaturated cycle which may contain up to four further heteroatoms selected from the group consisting of N, O and S, where two oxygen atoms are not adjacent,
R¹¹ are identical or different and represent C₁-C₈-alkyl, C₁-C₈-haloalkyl, C₁-C₄-trialkylsilyl, unsubstituted or substituted C₂-C₆-alkenyl, unsubstituted or substituted C₂-C₆-alkynyl, unsubstituted or substituted C₃-C₆-cycloalkyl, unsubstituted or substituted aryl, C₁-C₄-alkoxy-C₁-C₄-alkyl, unsubstituted or substituted benzyl or a 3- to 7-membered unsubstituted or substituted saturated or unsaturated cycle which may contain no or up to four heteroatoms selected from the group consisting of N, O and S, where two oxygen atoms are not adjacent,
where the substituents in R¹⁰ independently of one another are selected from the group consisting of:
methyl, ethyl, isopropyl, cyclopropyl, fluorine, chlorine and bromine atoms, methoxy, ethoxy, methylmercapto, ethylmercapto, cyano, hydroxyl and CF₃,
and agrochemically active salts thereof.

2. The Compound of formula (I) wherein
X¹ represents sulphur or CR¹,
X² represents sulphur or CR²,
where exactly one of the radicals X¹ and X² representsa sulphur atom,
R¹ represents hydrogen, methyl, methoxy or Cl,
if X² represents CR²,
R² and R³ independently of one another represent hydrogen, halogen, CN, nitro, hydroxyl, O—C₁-C₄-alkyl, O—(C₁-C₃-haloalkyl), O—(C₃-C₆-cycloalkyl), O—C₂-C₄-alkenyl, O—C₂-C₄-alkynyl, O(CH₂)ₘO(C₁-C₄-alkyl), OPh, OCO(C₁-C₄-alkyl), SH, S—C₁-C₄-alky, S—C₁-C₃-haloalkyl, SPh, SO(C₁-C₄-alkyl), SO₂(C₁-C₄-alkyl), SO₂(C₁-C₃-haloalkyl), SO₂(C₂-C₄-alkenyl), SO₂(C₂-C₄-alkynyl), CHO, CO(C₁-C₄-alkyl), CH═NO(C₁-C₄-alkyl), C(C₁-C₄-alkyl)=NO(C₁-C₄-alkyl), CONH(C₁-C₄-alkyl), CON(C₁-C₄-alkyl)₂, CON(SiMe₃)₂, CONH(C₁-C₃-haloalkyl), CONH(C₂-C₄-alkenyl), CONH(C₂-C₄-alkynyl), CONH(C₃-C₆-cycloalkyl), CONHCH₂C(═CH₂)CH₃, CONHCH(CH₃)CH₂O(C₁-C₄-alkyl), CONH(CH₂)ₘ, O(C₁-C₄-alkyl), CONH(CH₂)ₘS(C₁-C₄-alkyl), CONHCH(CH₃)CH₂S(C₁-C₄-alkyl), CONHPh, pyrrolidin-1-ylcarbonyl, piperidin-1-ylcarbonyl, (4-methylpiperazin-1-yl)carbonyl, azetidin-1-ylcarbonyl, aziridin-1-ylcarbonyl, hexamethylenimin-1-yl-carbonyl, morpholin-1-ylcarbonyl, thiomorpholin-1-ylcarbonyl, COOH, COCl, (C₁-C₄-alkoxy)carbonyl, NHCO(C₁-C₄-alkyl), NHCO(C₁-C₄-haloalkyl), N(C₁-C₂-alkyl)CO(C₁-C₄-alkyl), NHCO(C₂-C₄-alkenyl), NHCOPh, NHCO(C═CH₂)CH₃, NHCON(C₁-C₄-alkyl)₂, NHCO(CH₂)ₘO(C₁-C₄-alkyl), NHCHO, N(C₁-C₄-alkyl)CHO, NH₂, NH(C₁-C₄-alkyl), N(C₁-C₄-alkyl)₂, NHCH(C₁-C₄-alkyl)CH₂O(C₁-C₄-alkyl), (CH₂)ₘCN, (CH₂)ₘSO(C₁-C₄-alkyl), (CH₂)ₘSO₂(C₁-C₄-alkyl), (CH₂)ₘCO(C₁-C₄-alkyl), (CH₂)ₘO(C₁-C₄-alkyl), C(CH₃)₂O(C₁-C₄-alkyl), (CH₂)ₘC(C₁-C₄-alkyl)₂O(C₁-C₄-alkyl), CH₂OH, (CH₂)ₘS(C₁-C₄-alkyl), C(CH₃)₂S(C₁-C₄-alkyl), (CH₂)ₘNH(C₁-C₄-alkyl), (CH₂)ₘN(C₁-C₄-alkyl)₂, C₁-C₅-alkyl, C₃-C₆-cycloalkyl or C₁-C₃-haloalkyl,
where, if X² represents a sulphur atom, the above definition applies only to R³ and m corresponds to a number from 1 to 4,
R⁴ represents hydrogen, methyl, methoxy, chlorine or fluorine,
R⁵ represents hydrogen, Me, COMe, CHO, COCH₂OCH₃, CH₂OCH₃, COOMe, COOEt, COOtertBu, COOBn, COCF₃, CH₂CH═CH₂, CH₂CCH, SOCH₃, SO₂CH₃ or benzyl,
R⁶ represents hydrogen, cyano, methyl, CF₃ or CFH₂,
R⁷ represents fluorine, chlorine, bromine, iodine, methyl, OCF₃ or CF₃,
R⁸ represents hydrogen, methyl, ethyl, propyl, propan-2-yl, 2-methoxyethan-1-yl, prop-2-en-1-yl, CH₂OCH₃, COH, COMe, COOMe, COOEt, COOtertBu, COCF₃ or benzyl,
R⁹ represents straight-chain or branched unsubstituted or substituted C₁-C₅-alkyl, unsubstituted or substituted C₃-C₆-cycloalkyl, straight-chain or branched unsubstituted or substituted C₃-C₆-cycloalkyl-C₁-C₂-alkyl, unsubstituted or substituted C₂-C₅-haloalkyl, straight-chain or branched unsubstituted or substituted C₃-C₅-alkenyl or 2-methyl-1-(methylsulphanyl)propan-2-yl,
where the substituents in R⁹ independently of one another are selected from the group consisting of methyl, ethyl, isopropyl, cyclopropyl, fluorine, chlorine and bromine atoms, methoxy, ethoxy, methylmercapto, ethylmercapto, cyano, hydroxyl and CF₃,
and agrochemically active salts thereof.

3. The compound of formula (I) wherein:
X¹ represents sulphur or CR¹,
X² represents sulphur or CR²,
where exactly one of the radicals X¹ and X² is a sulphur atom,
R¹ represents hydrogen,
if X² represents CR²,
R² and R³ independently of one another represent hydrogen, COOMe, COOEt, COOPr, COOiPr, CONH(C₄H₉), CONH(CH₂)₂OMe, CONHCH(CH₃)CH₂OMe, CONHOH, CONHMe, CONHEt, CONHPr, CONHiPr, CONH(i-C₄H₉), CONHPh, CONH(CH₂)₂SCH₃, CONHCH(CH₃)CH₂SCH₃, CONHCH₂CF₃, CONHCH₂CH═CH₂, CONHCH₂C≡CH, CONMeCH₂C≡CH, CONHCH₂C(═CH₂)CH₃, CONHPh, CONHcyclopropyl, CON(Me)iC₃H₇, CON(Me)CH₂CH═CH₂, CON(Et)₂, CON(Me)cyclopropylmethyl, CON(Me)cyclobutylmethyl, CON(Ph)₂, CON(Me)₂, CON(Pr)₂, pyrrolidin-1-ylcarbonyl, piperidin-1-ylcarbonyl, (4-methylpiperazin-1-yl)carbonyl, azetidin-1-ylcarbonyl, aziridin-1-yl-carbonyl, hexamethylenimin-1-ylcarbonyl, morpholin-1-ylcarbonyl, thiomorpholin-1-ylcarbonyl, CON(SiMe₃)₂, COMe, COEt, COPr, CN, C(═NOCH₃)Me, C(═NOEt)Me, C(═NOPr)Me, chlorine, bromine, iodine, nitro, SH, SMe, SEt, SPr, SCF₃, SPh, COOH, Me, Et, Pr, SO₂Me, SO₂Et, CH₂OMe or CH₂OEt,
where, if X² represents a sulphur atom, the above definition applies only to R³,
R⁴ represents hydrogen,
R⁵ represents hydrogen, COMe, CHO, CH₂OCH₃, COOMe or CH₂C≡CH,
R⁶ represents hydrogen,
R⁷ represents fluorine, chlorine, bromine, iodine, OCF₃ or CF₃,
R⁸ represents hydrogen or methyl,
R⁹ represents cyclopropyl, cyclobutyl, 2-methylcycloprop-1-yl, 2-methylcyclobut-1-yl, 3-methylcyclobut-1-yl, 2,2-difluoroethyl, 2,2,2-trifluoroethyl, isopropyl, cyclopropylmethyl, methyl, ethyl, 2,2-dimethylcyclopropyl, cyclopentyl, propan-2-yl, prop-2-en-1-yl, butan-2-yl, 1-methoxypropan-2-yl, 2-methyl-1-(methylsulphanyl)propan-2-y oxetan-3-yl, 1,1,1-trifluoropropan-2-yl or 2,2,3,3,3-pentafluoropropyl,
and agrochemically active salts thereof.

4. The compound of formula (I) wherein
X¹ represents sulphur or CR¹
X² represents sulphur or CR²,
where exactly one of the radicals X¹ and X² represents a sulphur atom,
R¹ represents hydrogen,
if X² represents CR²,
R² and R³ independently of one another represent hydrogen, CH₃, COOMe, CONH(tert-C₄H₉), CONH(CH₂)₂OMe, CONHCH(CH₃)CH₂OMe, CONHOH, CON(Me)iC₃H₇, CON(Me)CH₂CH═CH₂, CON(Et)₂, CONmethylcyclopropylmethyl, pyrrolidin-1-ylcarbonyl, piperidin-1-ylcarbonyl, morpholin-1-ylcarbonyl, COMe, CN, C(═NOCH₃)CH₃, chlorine, bromine, SMe, CONHCH₂CF₃, CONHCH₂CH═CH₂, CONHCH₂CεCH, CONMeCH₂C≡CH, CONHCH₂C(═CH₂)CH₃, CONHPh, CONHcyclopropyl, (4-methylpiperazin-1-yl)carbonyl, COOH or SO₂Me,
where, if X² represents a sulphur atom, the above definition applies only to R³,
R⁴ represents hydrogen,
R⁵ represents hydrogen,
R⁶ represents hydrogen,
R⁷ represents chlorine, bromine or CF₃,
R⁸ represents hydrogen,
R⁹ represents cyclopropyl, cyclobutyl, 2-methylcycloprop-1-yl, 2-methylcyclobut-1-yl, 3-methylcyclobut-1-yl, 2,2-difluoroethyl, 2,2,2-trifluoroethyl, isopropyl or cyclopropylmethyl,
and agrochemically active salts thereof.

5. A composition for controlling phytopathogenic harmful fungi, comprising at least one compound of formula (I) according to claim 1, and one or more extenders surfactants or a combination thereof.

6. (canceled)

7. A method for controlling phytopathogenic harmful fungi, comprising applying the compound of formula (I) according to claim 1 to one or more phytopathogenic harmful fungi, their habitat or a combination thereof.

8. A process for preparing the composition for controlling phytopathogenic harmful fungi, comprising mixing the compound of formula (I) according to claim 1 with one or more extenders, surfactants or a combination thereof.

9. A process for preparing the compound of formula (I) according to the invention, comprising at least one of steps (a) to (e) below:
(a) reacting a compound of formula (III) with a compound of the formula (II) in the presence of a base, if appropriate in the presence of a solvent, if appropriate in the presence of a catalyst, to give a compound of formula (V), according to the reaction scheme below: 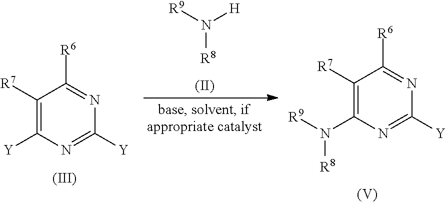
where Y═F, Cl, Br or I,
(b) reacting the compound of formula (V) with a compound of formula (IV), if appropriate in the presence of an acid, if appropriate in the presence of a solvent, according to the reaction scheme below: 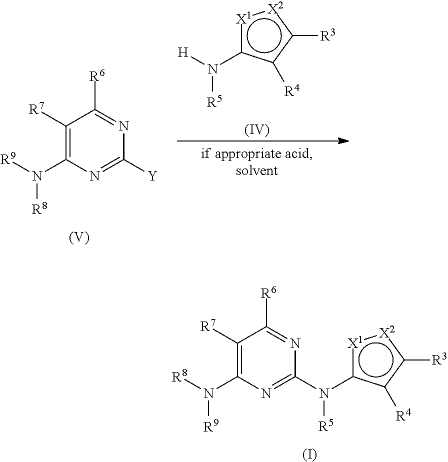
where Y═F, Cl, Br or I,
(c) reacting a compound of the formula (VI) with the compound of formula (IV), if appropriate in the presence of an acid and in the presence of a solvent, according to the reaction scheme below: 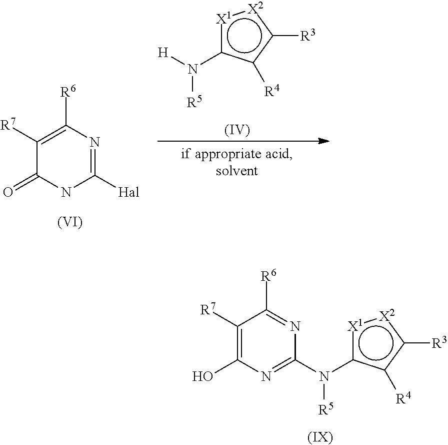
where Hal=F, Cl, Br or I,
(d) reacting a compound of formula (IX) with a halogenating agent, if appropriate in the presence of a solvent, to give a compound of formula (X), according to the reaction scheme below: 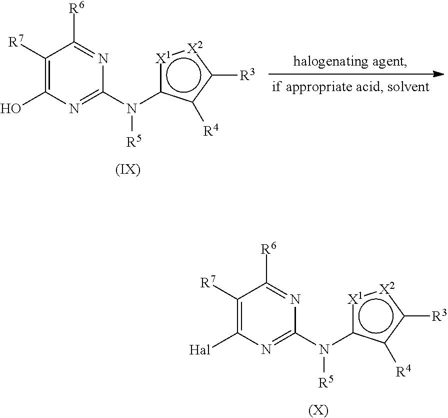
(e) reacting the compound of formula (X) with the compound of formula (II) in the presence of a base, if appropriate in the presence of a solvent, if appropriate in the presence of a catalyst, to give the compound of the formula (I), according to the reaction scheme below:

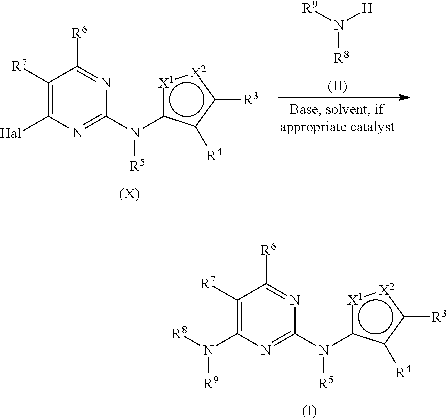

10. The compound of formula (IX) 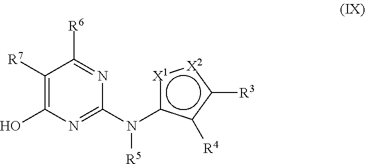
wherein
X¹ and X² to R⁷ correspond to the definitions according to claim 1.

11. The compound of the formula (X) 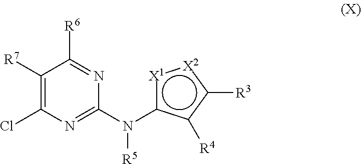
wherein
X¹, X², and R¹ to R⁷ correspond to the definitions according to claim 1.

12. The compound of formula (V) 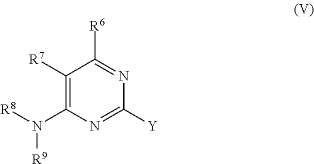
wherein
R⁶ represents hydrogen,
and, if
R⁷ represents I, SMe, SOMe, SO₂Me, CF₃, CFH₂ or CF₂H, and
Y represents F, Cl, Br or I,
R⁸ represents hydrogen, ethyl, propyl, propan-2-yl, 2-methoxyethan-1-yl, prop-2-en-1-yl, CH₂OCH₃, COMe, COOMe, COOEt, COOtertBu, COCF₃ or benzyl, and
R⁹ represents cyclopropyl, cyclobutyl, 2-methylcycloprop-1-yl, 2-methylcyclobut-1-yl, 3-methylcyclobut-1-yl, 2,2-difluoroethyl, 2,2,2-trifluoroethyl, isopropyl, cyclopropylmethyl, methyl, ethyl, 2,2-dimethylcyclopropyl, cyclopentyl, propan-2-yl, prop-2-en-1-yl, butan-2-yl, 1-methoxypropan-2-yl, 2-methyl-1-(methylsulphanyl)propan-2-yl, 1,1,1-trifluoropropan-2-yl or 2,2,3,3,3-pentafluoropropyl,
and, if
R⁷ represents cyano, and
Y represents F, Cl, Br or I,
R⁸ represents hydrogen, methyl, propyl, propan-2-yl, 2-methoxyethan-1-yl, prop-2-en-1-yl, CH₂OCH₃, COMe, COOMe, COOEt, COOtertBu, COCF₃ or benzyl and
R⁹ represents cyclobutyl, 2-methylcycloprop-1-yl, 2-methylcyclobut-1-yl, 3-methylcyclobut-1-yl, 2,2-difluoroethyl, isopropyl, cyclopropylmethyl, 2,2-dimethylcyclopropyl, cyclopentyl, butan-2-yl, 1-methoxypropan-2-yl, 2-methyl-1-(methylsulphanyl)propan-2-yl, 1,1,1-trifluoropropan-2-yl or 2,2,3,3,3-pentafluoropropyl.

13. A composition for controlling phytopathogenic harmful fungi, comprising at least one compound of formula (I) according to claim 2, and one or more extenders, surfactants or a combination thereof.

14. A composition for controlling phytopathogenic harmful fungi, comprising at least one compound of formula (I) according to claim 3, and one or more extenders, surfactants or a combination thereof.

15. A composition for controlling phytopathogenic harmful fungi, comprising at least one compound of formula (I) according to claim 4, and one or more extenders, surfactants or a combination thereof.

16. A method for controlling phytopathogenic harmful fungi, comprising applying the compound of formula (I) according to claim 2 to one or more phytopathogenic harmful fungi, their habitat or a combination thereof.

17. A method for controlling phytopathogenic harmful fungi, comprising applying the compound of formula (I) according to claim 3 to one or more phytopathogenic harmful fungi, their habitat or a combination thereof.

18. A method for controlling phytopathogenic harmful fungi, comprising applying the compound of formula (I) according to claim 4 to one or more phytopathogenic harmful fungi, their habitat or a combination thereof.

19. A process for preparing the composition for controlling phytopathogenic harmful fungi, comprising mixing the compound of formula (I) according to claim 2 with one or more extenders, surfactants or a combination thereof.

20. A process for preparing the composition for controlling phytopathogenic harmful fungi, comprising mixing the compound of formula (I) according to claim 3 with one or more extenders, surfactants or a combination thereof.

21. A process for preparing the composition for controlling phytopathogenic harmful fungi, comprising mixing the compound of formula (I) according to claim 4 with one or more extenders, surfactants or a combination thereof.
